SEQUENCE LISTING (39 sequences)

SEQ ID NO: 1 - protein, 288 aa, Homo sapiens
MGHTRRQGTSPSKCPYLNFFQLLVLAGLSHFCSGVIHVTKEVKEVATLSCGHNVSVEELAQTRIYWQKEKKMVLTMMSGDMNIWPEYKNRTIFDITNNLSIVILALRPSDEGTYECVVLKYEKDAFKREHLAEVTLSVKADFPTPSISDFEIPTSNIRRIICSTSGGFPEPHLSWLENGEELNAINTTVSQDPETELYAVSSKLDFNMTTNHSFMCLIKYGHLRVNQTFNWNTTKQEHFPDNLLPSWAITLISVNGIFVICCLTYCFAPRCRERRRNERLRRESVRPV

SEQ ID NO: 2 - DNA, 972 bp, Homo sapiens
atgggactgagtaacattctctttgtgatggccttcctgctctctggtgctgctcctctgaagattcaagcttatttcaatgagactgcagacctgccatgccaatttgcaaactctcaaaaccaaagcctgagtgagctagtagtattttggcaggaccaggaaaacttggttctgaatgaggtatacttaggcaaagagaaatttgacagtgttcattccaagtatatgggccgcacaagttttgattcggacagttggaccctgagacttcacaatcttcagatcaaggacaagggcttgtatcaatgtatcatccatcacaaaaagcccacaggaatgattcgcatccaccagatgaattctgaactgtcagtgcttgctaacttcagtcaacctgaaatagtaccaatttctaatataacagaaaatgtgtacataaatttgacctgctcatctatacacggttacccagaacctaagaagatgagtgttttgctaagaaccaagaattcaactatcgagtatgatggtattatgcagaaatctcaagataatgtcacagaactgtacgacgtttccatcagcttgtctgtttcattccctgatgttacgagcaatatgaccatcttctgtattctggaaactgacaagacgcggcttttatcttcacctttctctatagagcttgaggaccctcagcctcccccagaccacattccttggattacagctgtacttccaacagttattatatgtgtgatggttttctgtctaattctatggaaatggaagaagaagaagcggcctcgcaactcttataaatgtggaaccaacacaatggagagggaagagagtgaacagaccaagaaaagagaaaaaatccatatacctgaaagatctgatgaagcccagcgtgtttttaaaagttcgaagacatcttcatgcgacaaaagtgatacatgtttttaa

SEQ ID NO: 3 - protein, 323 aa, Homo sapiens
MGLSNILFVMAFLLSGAAPLKIQAYFNETADLPCQFANSQNQSLSELVVFWQDQENLVLNEVYLGKEKFDSVHSKYMGRTSFDSDSWTLRLHNLQIKDKGLYQCIIHHKKPTGMIRIHQMNSELSVLANFSQPEIVPISNITENVYINLTCSSIHGYPEPKKMSVLLRTKNSTIEYDGIMQKSQDNVTELYDVSISLSVSFPDVTSNMTIFCILETDKTRLLSSPFSIELEDPQPPPDHIPWITAVLPTVIICVMVFCLILWKWKKKKRPRNSYKCGTNTMEREESEQTKKREKIHIPERSDEAQRVFKSSKTSSCDKSDTCF

SEQ ID NO: 4 - DNA, 834 bp, Homo sapiens
atggttcgtctgcctctgcagtgcgtcctctggggctgcttgctgaccgctgtccatccagaaccacccactgcatgcagagaaaaacagtacctaataaacagtcagtgctgttctttgtgccagccaggacagaaactggtgagtgactgcacagagttcactgaaacggaatgccttccttgcggtgaaagcgaattcctagacacctggaacagagagacacactgccaccagcacaaatactgcgaccccaacctagggcttcgggtccagcagaagggcacctcagaaacagacaccatctgcacctgtgaagaaggctggcactgtacgagtgaggcctgtgagagctgtgtcctgcaccgctcatgctcgcccggctttggggtcaagcagattgctacaggggtttctgataccatctgcgagccctgcccagtcggcttcttctccaatgtgtcatctgctttcgaaaaatgtcacccttggacaagctgtgagaccaaagacctggttgtgcaacaggcaggcacaaacaagactgatgttgtctgtggtccccaggatcggctgagagccctggtggtgatccccatcatcttcgggatcctgtttgccatcctcttggtgctggtctttatcaaaaaggtggccaagaagccaaccaataaggccccccaccccaagcaggaaccccaggagatcaattttcccgacgatcttcctggctccaacactgctgctccagtgcaggagactttacatggatgccaaccggtcacccaggaggatggcaaagagagtcgcatctcagtgcaggagagacagtga

SEQ ID NO: 5 - protein, 277 aa, Homo sapiens
MVRLPLQCVLWGCLLTAVHPEPPTACREKQYLINSQCCSLCQPGQKLVSDCTEFTETECLPCGESEFLDTWNRETHCHQHKYCDPNLGLRVQQKGTSETDTICTCEEGWHCTSEACESCVLHRSCSPGFGVKQIATGVSDTICEPCPVGFFSNVSSAFEKCHPWTSCETKDLVVQQAGTNKTDVVCGPQDRLRALVVIPIIFGILFAILLVLVFIKKVAKKPTNKAPHPKQEPQEINFPDDLPGSNTAAPVQETLHGCQPVTQEDGKESRISVQERQ

SEQ ID NO: 6 - protein, 735 aa, Homo sapiens
MVVILGASNILWIMFAASQAFKIETTPESRYLAQIGDSVSLTCSTTGCESPFFSWRTQIDSPLNGKVTNEGTTSTLTMNPVSFGNEHSYLCTATCESRKLEKGIQVEIYSFPKDPEIHLSGPLEAGKPITVKCSVADVYPFDRLEIDLLKGDHLMKSQEFLEDADRKSLETKSLEVTFTPVIEDIGKVLVCRAKLHIDEMDSVPTVRQAVKELQVYISPKNTVISVNPSTKLQEGGSVTMTCSSEGLPAPEIFWSKKLDNGNLQHLSGNATLTLIAMRMEDSGIYVCEGVNLIGKNRKEVELIVQEKPFTVEISPGPRIAAQIGDSVMLTCSVMGCESPSFSWRTQIDSPLSGKVRSEGTNSTLTLSPVSFENEHSYLCTVTCGHKKLEKGIQGELYSFPRDPEIEMSGGLVNGSSCTVSCKVPSVYPLDRLEIELLKGETILENIEFLEDTDMKSLENKSLEMTFIPTIEDTGKALVCQAKLHIDDMEFEPKQRQSTQTLYVNVAPRDTTVLVSPSSILEEGSSVNMTCLSQGFPAPKILWSRQLPNGELQPLSENATLTLISTKMEDSGVYLCEGINQAGRSRKEVELIIQVTPKDIKLTAFPSESVKEGDTVIISCTCGNVPETWIILKKKAETGDTVLKSIDGAYTIRKAQLKDAGVYECESKNKVGSQLRSLTLDVQGRENNKDYFSPELLVLYFASSLIIPAIGMIIYFARKANMKGSYSLVEAQKSKV

SEQ ID NO: 7 - DNA, 945 bp, Mus musculus
atgttctccaaagcatctgaagctatggcttgcaattgtcagttgatgcaggatacaccactcctcaagtttccatgtccaaggctcattcttctctttgtgctgctgattcgtctttcacaagtgtcttcagatgttgatgaacaactgtccaagtcagtgaaagataaggtattgctgccttgccgttacaactctcctcatgaagatgagtctgaagaccgaatctactggcaaaaacatgacaaagtggtgctgtctgtcattgctgggaaactaaaagtgtggcccgagtataagaaccggactttatatgacaacactacctactctcttatcatcctgggcctggtcctttcagaccggggcacatacagctgtgtcgttcaaaagaaggaaagaggaacgtatgaagttaaacacttggctttagtaaagttgtccatcaaagctgacttctctacccccaacataactgagtctggaaacccatctgcagacactaaaaggattacctgctttgcttccgggggtttcccaaagcctcgcttctcttggttggaaaatggaagagaattacctggcatcaatacgacaatttcccaggatcctgaatctgaattgtacaccattagtagccaactagatttcaatacgactcgcaaccacaccattaagtgtctcattaaatatggagatgctcacgtgtcagaggacttcacctgggaaaaacccccagaagaccctcctgatagcaagaacacacttgtgctctttggggcaggattcggcgcagtaataacagtcgtcgtcatcgttgtcatcatcaaatgcttctgtaagcacagaagctgtttcagaagaaatgaggcaagcagagaaacaaacaacagccttaccttcgggcctgaagaagcattagctgaacagaccgtcttcctttag

SEQ ID NO: 8 - protein, 314 aa, Mus musculus
MFSKASEAMACNCQLMQDTPLLKFPCPRLILLFVLLIRLSQVSSDVDEQLSKSVKDKVLLPCRYNSPHEDESEDRIYWQKHDKVVLSVIAGKLKVWPEYKNRTLYDNTTYSLIILGLVLSDRGTYSCVVQKKERGTYEVKHLALVKLSIKADFSTPNITESGNPSADTKRITCFASGGFPKPRFSWLENGRELPGINTTISQDPESELYTISSQLDFNTTRNHTIKCLIKYGDAHVSEDFTWEKPPEDPPDSKNTLVLFGAGFGAVITVVVIVVIIKCFCKHRSCFRRNEASRETNNSLTFGPEEALAEQTVFL

SEQ ID NO: 9 - DNA, 930 bp, Mus musculus
atggaccccagatgcaccatgggcttggcaatccttatctttgtgacagtcttgctgatctcagatgctgtttccgtggagacgcaagcttatttcaatgggactgcatatctgccgtgcccatttacaaaggctcaaaacataagcctgagtgagctggtagtattttggcaggaccagcaaaagttggttctgtacgagcactatttgggcacagagaaacttgatagtgtgaatgccaagtacctgggccgcacgagctttgacaggaacaactggactctacgacttcacaatgttcagatcaaggacatgggctcgtatgattgttttatacaaaaaaagccacccacaggatcaattatcctccaacagacattaacagaactgtcagtgatcgccaacttcagtgaacctgaaataaaactggctcagaatgtaacaggaaattctggcataaatttgacctgcacgtctaagcaaggtcacccgaaacctaagaagatgtattttctgataactaattcaactaatgagtatggtgataacatgcagatatcacaagataatgtcacagaactgttcagtatctccaacagcctctctctttcattcccggatggtgtgtggcatatgaccgttgtgtgtgttctggaaacggagtcaatgaagatttcctccaaacctctcaatttcactcaagagtttccatctcctcaaacgtattggaaggagattacagcttcagttactgtggccctcctccttgtgatgctgctcatcattgtatgtcacaagaagccgaatcagcctagcaggcccagcaacacagcctctaagttagagcgggatagtaacgctgacagagagactatcaacctgaaggaacttgaaccccaaattgcttcagcaaaaccaaatgcagagtga

SEQ ID NO: 10 - protein, 309 aa, Mus musculus
MDPRCTMGLAILIFVTVLLISDAVSVETQAYFNGTAYLPCPFTKAQNISLSELVVFWQDQQKLVLYEHYLGTEKLDSVNAKYLGRTSFDRNNWTLRLHNVQIKDMGSYDCFIQKKPPTGSIILQQTLTELSVIANFSEPEIKLAQNVTGNSGINLTCTSKQGHPKPKKMYFLITNSTNEYGDNMQISQDNVTELFSISNSLSLSFPDGVWHMTVVCVLETESMKISSKPLNFTQEFPSPQTYWKEITASVTVALLLVMLLIIVCHKKPNQPSRPSNTASKLERDSNADRETINLKELEPQIASAKPNAE

SEQ ID NO: 11 - DNA, 870 bp, Mus musculus
atggtgtctttgcctcggctgtgcgcgctatggggctgcttgttgacagcggtccatctagggcagtgtgttacgtgcagtgacaaacagtacctccacgatggccagtgctgtgatttgtgccagccaggaagccgactgacaagccactgcacagctcttgagaagacccaatgccacccatgtgactcaggcgaattctcagcccagtggaacagggagattcgctgtcaccagcacagacactgtgaacccaatcaagggcttcgggttaagaaggagggcaccgcagaatcagacactgtctgtacctgtaaggaaggacaacactgcaccagcaaggattgcgaggcatgtgctcagcacacgccctgtatccctggctttggagttatggagatggccactgagaccactgataccgtctgtcatccctgcccagtcggcttcttctccaatcagtcatcacttttcgaaaagtgttatccctggacaagctgtgaggataagaacttggaggtcctacagaaaggaacgagtcagactaatgtcatctgtggtttaaagtcccggatgcgagccctgctggtcattcctgtcgtgatgggcatcctcatcaccattttcggggtgtttctctatatcaaaaaggtggtcaagaaaccaaaggataatgagatgttaccccctgcggctcgacggcaagatccccaggagatggaagattatcccggtcataacaccgctgctccagtgcaggagacactgcacgggtgtcagcctgtcacacaggaggatggtaaagagagtcgcatctcagtgcaggagcggcaggtgacagacagcatagccttgaggcccctggtctga

SEQ ID NO: 12 - protein, 289 aa, Mus musculus
MVSLPRLCALWGCLLTAVHLGQCVTCSDKQYLHDGQCCDLCQPGSRLTSHCTALEKTQCHPCDSGEFSAQWNREIRCHQHRHCEPNQGLRVKKEGTAESDTVCTCKEGQHCTSKDCEACAQHTPCIPGFGVMEMATETTDTVCHPCPVGFFSNQSSLFEKCYPWTSCEDKNLEVLQKGTSQTNVICGLKSRMRALLVIPVVMGILITIFGVFLYIKKVVKKPKDNEMLPPAARRQDPQEMEDYPGHNTAAPVQETLHGCQPVTQEDGKESRISVQERQVTDSIALRPLV

SEQ ID NO: 13 - DNA, 994 bp, Porcus spp
atgggactgagtaacattctctttgtgatggtcctcctgctctctggtgctgcctccttgaaaagtcaggcatatttcaatgagactggagaactgccgtgccattttacaaactcgcagaacctaagcctggatgagctggtcatattttggcaggaccaggataacctggttctctacgagctataccgaggccaagagaagcctcataatgttaattccaagtatatgggtcgcacaagctttgaccaggccacctggaccctgagactccacaacgttcaaatcaaggacaagggctcatatcaatgtttcatccatcataaagggccgcatggacttgttcctatccaccagatgagttctgacctatcattgcttgctaacttcagtcaacctgaaataaacctacttactaatcacacagaaaattctgtcataaatttgacctgctcatctacacaaggctacccagaaccccagaggatgtatatgttgctaaatacgaagaattcaaccactgagcatgatgctgacatgaagaaatctcaaaataacatcacggaactctacaatgtatcaatcagggtgtctcttcccatccctcccgagacaaatgtgagcatcgtctgtgtcctgcaacttgagccaagcaagacactgcttttctccctaccttgtaatatagatgcaaagccacctgtgcaaccccctgtcccagaccacatcctctggattgcagctctacttgtaacagtggtcgttgtgtgtgggatggtgtcctttgtaacactaaggaaaaggaagaagaagcagcctggcccctctaatgaatgtggtgaaaccatcaaaatgaacaggaaggcgagtgaacaaactaagaacagagcagaagtccatgaacgatctgatgatgcccagtgtgatgttaatattttaaagacagcctcagatgacaacagtactacagatttttaattaaagagtaaactcc

SEQ ID NO: 14 - protein, 330 aa, Porcus spp
MGLSNILFVMVLLLSGAASLKSQAYFNETGELPCHFTNSQNLSLDELVIFWQDQDNLVLYELYRGQEKPHNVNSKYMGRTSFDQATWTLRLHNVQIKDKGSYQCFIHHKGPHGLVPIHQMSSDLSLLANFSQPEINLLTNHTENSVINLTCSSTQGYPEPQRMYMLLNTKNSTTEHDADMKKSQNNITELYNVSIRVSLPIPPETNVSIVCVLQLEPSKTLLFSLPCNIDAKPPVQPPVPDHILWIAALLVTVVVVCGMVSFVTLRKRKKKQPGPSNECGETIKMNRKASEQTKNRAEVHERSDDAQCDVNILKTASDDNSTTDFLKSKL

SEQ ID NO: 15 - DNA, 837 bp, Porcus
atggttcgtttgcctctgcagtgtctcctctggggctgctttttgaccgccgtccacccagaaccacccacttcatgcaaagaaaaccaatacccaacaaacagccggtgctgtaatttgtgcccgccaggacagaaactggtgaaccactgcacagaggtcactgaaacagaatgccttccttgcagttccagcgaattcctagccacctggaatagagagaaacactgtcatcagcacaaatactgcgaccccaacctaggtctccaggtccagagggagggcacctcgaaaacagacaccacttgtgtgtgcagtgaaggccatcactgtaccaacagcgcctgtgaaagttgcaccttgcacagcttgtgcttccctggcctcggggtcaagcagatggcgacagaggtttctgacactatctgtgaaccctgcccagttggcttcttctccaatgtatcatctgcttcagaaaagtgtcagccttggacaagctgcgagagcaaaggcctggtggaacaacgtgcggggactaacaagaccgatgttgtctgtggtttccagagtcggatgagagccctggtggttatccccatcacgctggggatcctgtttgccgtcctgttggtatttctctgtatcagaaaggtgaccaaggagcaggagactaaggccctgcaccctaagactgaaaggcaggatcccgtggagacgattgatctggaggattttcccgactccaccgctccggtgcaggagaccttacattggtgccagcccgtcacccaggaggacggcaaagagagtcgcatctcagtgcaggagagacagtga

SEQ ID NO: 16 - protein, 278 aa, Porcus
MVRLPLQCLLWGCFLTAVHPEPPTSCKENQYPTNSRCCNLCPPGQKLVNHCTEVTETECLPCSSSEFLATWNREKHCHQHKYCDPNLGLQVQREGTSKTDTTCVCSEGHHCTNSACESCTLHSLCFPGLGVKQMATEVSDTICEPCPVGFFSNVSSASEKCQPWTSCESKGLVEQRAGTNKTDVVCGFQSRMRALVVIPITLGILFAVLLVFLCIRKVTKEQETKALHPKTERQDPVETIDLEDFPDSTAPVQETLHWCQPVTQEDGKESRISVQERQ

SEQ ID NO: 17 - protein, 534 aa, Porcus
IVVIFGASNILWMVFAVSQNVKVEIFPEDKMIAQIGDSASLTCSAPDCESSLSFSWRTQIDSPLNGKVKTNGTRSTLVMNPVSFENEHSYLCTVSCGNLKGERGIQVEIYSFPKDPEIHWSSLPEVGKPVTVRCLVPDVYPVEKLEIELLKDNHSMVSQNFLELIDIKSKETKSLEFTFTPTEEDIGKAIVCQATLIIDGQPSVKTTPEKMQVYISPKDPVISVNPSTSLQEGDSMMMTCTSEGLPAPQISWSKKLDNGDQQLLSGNATLTLIAMRMEDSGIYVCEGVNPVGTNRKEVELTVQVAPRDTTISVNPSSTLEEGSSVNMTCSSDGFPAPKILWSKKLRDGNLEPLSENTTLTLTSTKMEDSGIYVCEGINQAGINRKEVELIIQAAPKDLQLTAFPSESVKEGDTVIISCTCGNVPPTLIILKKKAETGDTVLKSTDGAYTIHRARLADAGVYECESKNEIGLQLRSITLDVKGRESNKDYFSSELLVLYCASSLIIPAIGVIIYFARKANMRGSYSLVDAQKSKV

SEQ ID NO: 18 - DNA, 807 bp, Vacca spp
atggttcgtttgccactgcagtgtctcttctggggcttctttctgaccgccgtccactcagaaccagccactgcttgtggagagaagcaatacccagtgaacagtctttgctgtgatttgtgcccgccgggacagaaactggtgaacgactgcacagaggtcagcaaaacagaatgccagtcctgcggtaaaggcgaattcttgtccacctggaacagagagaaatactgtcacgagcacagatactgcaaccccaacctagggctccggatccagagcgagggtaccttgaatacagacaccatttgtgtatgtgtcgaaggccaacactgtaccagtcacacctgcgaaagttgcacgccccacagcttgtgtctccctggcttcggggtcaagcagatcgctacagggcttttggataccgtctgtgaaccctgcccgctcggcttcttctccaacgtgtcatctgcttttgaaaagtgtcaccgttggacaagctgcgagagaaaaggcctggtggaacaacacgtggggacgaacaagacagatgttgtctgcggtttccagagtcggatgaggaccctggtggtgatccccgtcacgatgggagtcttgtttgctgtcctgttggtatctgcctgtatcaggaacataaccaagaagcggcagctaaggccctgcaccctatggctgaaaggcaggatcccgtggagacgattgatccggaggattttcccggcccccacccgcctctccggtgcaagagaccttatgctggtgtcagccggtcgcccaggaggacggcaa

SEQ ID NO: 19 - protein, 269 aa, Vacca spp
MVRLPLQCLFWGFFLTAVHSEPATACGEKQYPVNSLCCDLCPPGQKLVNDCTEVSKTECQSCGKGEFLSTWNREKYCHEHRYCNPNLGLRIQSEGTLNTDTICVCVEGQHCTSHTCESCTPHSLCLPGFGVKQIATGLLDTVCEPCPLGFFSNVSSAFEKCHRWTSCERKGLVEQHVGTNKTDVVCGFQSRMRTLVVIPVTMGVLFAVLLVSACIRNITKKRQLRPCTLWLKGRIPWRRLIRRIFPAPTRLSGARDLMLVSAGRPGGRQ

SEQ ID NO: 20 - DNA, 867 bp, Vacca spp
atgggccacacacggaggcagggaacatcaccatccaagtgtccatacctcaatttctttcagctcttggtgctggctggtctttctcacttctgttcaggtgttatccacgtgaccaaggaagtgaaagaagtggcaacgctgtcctgtggtcacaatgtttctgttgaagagctggcacaaactcgcatctactggcaaaaggagaagaaaatggtgctgactatgatgtctggggacatgaatatatggcccgagtacaagaaccggaccatctttgatatcactaataacctctccattgtgatcctggctctgcgcccatctgacgagggcacatacgagtgtgttgttctgaagtatgaaaaagacgctttcaagcgggaacacctggctgaagtgacgttatcagtcaaagctgacttccctacacctagtatatctgactttgaaattccaacttctaatattagaaggataatttgctcaacctctggaggttttccagagcctcacctctcctggttggaaaatggagaagaattaaatgccatcaacacaacagtttcccaagatcctgaaactgagctctatgctgttagcagcaaactggatttcaatatgacaaccaaccacagcttcatgtgtctcatcaagtatggacatttaagagtgaatcagaccttcaactggaatacaaccaagcaagagcattttcctgataacctgctcccatcctgggccattaccttaatctcagtaaatggaatttttgtgatatgctgcctgacctactgctttgccccaagatgcagagagagaaggaggaatgagagattgagaagggaaagtgtacgccctgtataa

SEQ ID NO: 21 - DNA, 35 bp, Porcus spp
gcatggatccatgggactgagtaacattctctttg

SEQ ID NO: 22 - DNA, 34 bp, Porcus
gcatgtcgacttaaaaatctgtagtactgttgtc

SEQ ID NO: 23 - DNA, 17 bp, Porcus
agaccgtcttcctttag

SEQ ID NO: 24 - DNA, 21 bp, Porcus
ttggatcctccatgttatccc

SEQ ID NO: 25 - DNA, 12 bp, Porcus
agcatctgaagc

SEQ ID NO: 26 - DNA, 22 bp, Porcus spp
atggatcctccattttccaacc

SEQ ID NO: 27 - DNA, 18 bp, Porcus spp
ttgtcgacatctactggc

SEQ ID NO: 28 - DNA, 58 bp, Porcus spp
ggatcctcactgtctctcctgatgagatgcgactctcctctttgcccgtccgtcctcc

SEQ ID NO: 29 - DNA, 29 bp, Porcus spp
gaattcatggttctgttgcctctgcagtg

SEQ ID NO: 30 - protein, 27 aa, Artificial Sequence
  Description of Artificial Sequence Porcus spp/ovalbumen chimeric peptide
ISQAVHAAHAEINEAGRSFDQATWTLR

SEQ ID NO: 31 - protein, 26 aa, Artificial Sequence
  Description of Artificial SequencePorcus spp/ovalbumen chimeric peptide
ISQAVHAAHAEINEAGRLPCHFTNSQ

SEQ ID NO: 32 - protein, 30 aa, Artificial Sequence
  Description of Artificial SequencePorcus spp/ovalbumen chimeric peptide
ISQAVHAAHAEINEAGRKGPHGLVPIHQMS

SEQ ID NO: 33 - protein, 26 aa, Artificial Sequence
  Description of Artificial Sequence Porcus spp/ovalbumen chimeric peptide
ISQAVHAAHAEINEAGRGLVPIHQMS

SEQ ID NO: 34 - protein, 28 aa, Artificial Sequence
  Description of Artificial Sequence Porcus spp/ovalbumen chimeric peptide
ISQAVHAAHAEINEAGRVQIKDKGSYQC

SEQ ID NO: 35 - protein, 29 aa, Artificial Sequence
  Description of Artificial Sequence Porcus spp/ovalbumen chimeric peptide
ISQAVHAAHAEINEAGRCSSTQGYPEPQR

SEQ ID NO: 36 - protein, 29 aa, Artificial Sequence
  Description of Artificial Sequence Porcus spp/ovalbumen chimeric peptide
ISQAVHAAHAEINEAGRKSQAYFNETGEL

SEQ ID NO: 37 - protein, 29 aa, Artificial Sequence
  Description of Artificial Sequence Porcus spp/ovalbumen chimeric peptide
ISQAVHAAHAEINEAGRASLKSQAYFNET

SEQ ID NO: 38 - protein, 30 aa, Artificial Sequence
  Description of Artificial Sequence Porcus spp/ovalbumen chimeric peptide
ISQAVHAAHAEINEAGRYMGRTSFDQATWT

SEQ ID NO: 39 - protein, 17 aa, Artificial Sequence
  Description of Artificial Sequence Porcus spp/ovalbumen chimeric peptide
ISQAVHAAHAEINEAGR

(19) United States
(12) Patent Application Publication
(10) Pub. No.: US 2006/0134124 A1
(43) Pub. Date: Jun. 22, 2006

(54) Immunosuppression

(75) Inventors: Robert Ian Lechler, London (GB); Nichola Jane Rogers, London (GB); Anthony Dorling, London (GB)

Correspondence Address: KLARQUIST SPARKMAN, LLP, 121 SW SALMON STREET, SUITE 1600, PORTLAND, OR, 97204, US

(73) Assignee: ML Laboratories PLC

(21) Appl. No.: 11/170,797

(22) Filed: Jun. 28, 2005

Related U.S. Application Data
(63) Continuation of application No. 09/868,605, filed on Sep. 12, 2001, now abandoned.

(30) Foreign Application Priority Data
Oct. 23, 1999 (GB) 9925015.1
Dec. 19, 1998 (GB) 9827921.9

(51) Int. Cl. A61K 39/00

(52) U.S. Cl. 424/184.1; 424/185.1

(57) ABSTRACT

The invention herein described relates to a method to improve the tolerance of a mammal, preferably a human, to a xenograft through immunisation of the recipient mammal with an immunogen comprising both a B cell epitope derived from porcine polypetides and T cell epitope. The invention also encompasses immunogenic compositions comprising said immunogens and methods to monitor the status of the xenograft.

### 1. FIELD OF THE INVENTION

This invention relates to immunosuppression and, more particularly, to immunosuppression in the context of xenotransplantation.

### 2. BACKGROUND TO THE INVENTION

Despite the established success of allogeneic organ transplantation, the increasing disparity between the supply and demand of organs must be overcome. Increasing the supply of allogeneic organs does not offer a satisfactory solution because even if all usable organs were transplanted this would still not meet the existing demand (1, 2). This has led to a resurgence of interest in xenotransplantation (the transplantation of organs between animals of different species) as a viable and attractive alternative.

Xenotransplantation research has recently focused on the pig as a suitable animal donor in terms of size, physiological compatibility and breeding characteristics (3, 4). Until recently however, discordant xenotransplantation has been limited by the inevitable occurrence of humorally-mediated hyperacute rejection (HAR) which rapidly triggers organ rejection upon revascularisation. HAR is the fate of most organs transplanted between discordant species. Recently, significant advances have been made in understanding the immunological basis of HAR, and many approaches have been employed to overcome it. Of significance, a variety of transgenic strategies are currently being employed including the expression of regulators of complement activity on porcine endothelial cells (5). It is foreseeable that short-term xenograft survival will soon be achieved (6). The recent advances in overcoming HAR have highlighted subsequent immunological barriers which must be surmounted to enable long-term xenograft survival. Both humoral and cellular arms of the immune response appear to play a role in the downstream events of immunological rejection. Clearly the most important of which is the existence of a formidable T cell mediated rejection response (7-11) previously obscured by the dominant role of HAR. In vitro, human T cells have been demonstrated to play a central role in the recognition of xenogeneic cells (7, 8, 12) following sensitisation via the direct and indirect T cell activation pathways, which have been well documented for allorecognition and allograft rejection (13). Knowledge of the cellular mechanisms underlying allorejection has provided an important basis for the investigation of the T cell mediated xenoresponse.

At present, the major therapies to prevent cell mediated rejection of organ transplants rely on systemic immunonosuppressive drugs or monoclonal antibody (Mab) therapy directed against targets such as CD3, CD4, CD25, (14). Following reports that strong T cell xenoresponses can be generated in vitro (7, 8, 12), control of xenograft rejection may require levels of immunosuppresion much greater than the current standard doses. Such a strategy would not be desired in a xenograft context. Drugs must be taken for life, depress the entire immune system and result in an increased risk of infection and susceptibility to cancer (14). For the applicability of xenotransplantation to the clinic, targeting graft-specific strategies for tolerance induction/immunosuppression would clearly be highly advantageous. Whilst this has been difficult to achieve in an allotransplant context, xenotransplantation offers greater potential—with differences between species providing the option for the generation of reagents that are truly graft specific. In addition, there is the opportunity for the manipulation of both the porcine donor organ, and the human recipient's immune system, prior to transplantation (1).

### 3. DETAILED BACKGROUND

3.1 T Cell Activation and Proliferation

Optimal proliferation of T cells, although initiated via ligation of the antigen specific CD3/TCR complex (Signal 1) requires additional costimulatory signals (Signal 2) (15, 16, 17) which are usually supplied by the antigen presenting cell (APC). Whilst antigenic stimulation of T cells in the presence of signal 2 induces T cell activation and proliferation (18), exposure of T cells to MHC-antigen complexes in their absence leads to aborted T cell proliferation and the development of clonal anergy (19, 20). Manipulation of APC by aldehyde fixation (20, 21) or heat treatment (19) has been demonstrated to abrogate the ability of such cells to activate alloreactive T cells, without altering levels of MHC-II surface expression. Thus T cell receptor occupancy alone is insufficient to fully activate the T cell (17). Anergic T cells are best characterised by their lack of IL-2 production and their continued inability to produce IL-2 on subsequent exposure to antigen (22). Thus, confirming the two signal model of activation as predicted by Lafferty et al (23). For T cells to respond to a given antigenic stimulus, multiple activation signals are required from the APC (23).

The in vivo induction of T cell anergy in the absence of a secondary signal was first demonstrated by Jenkins and Schwartz in 1986 (24) using chemically fixed APC to present specific peptide to CD4 T helper clones. A multitude of in vitro and in vivo data has since been produced supporting the hypothesis that signal 1 in isolation fails to activate T cells (22), and that costimulatory signalling results from contact with other cells rather than via soluble factors. Fibroblasts transfected with human Class II MHC molecules, but not expressing the appropriate CS signals (lacking signal 2) can efficiently present antigen to class II restricted CD4 T cell clones, but these fail to cause antigen specific T cell proliferation, rendering cells anergic. The context in which T cells first encounter antigen therefore has an important bearing on subsequent immune responsiveness.

Thus, costimulatory molecules are essential for T cell activation and multiplication and result from interactions between receptors on T cells and their ligands expressed on the APC. The costimulatory signal itself, however, is neither antigen specific nor MHC restricted (25). In recent years the molecular interactions involved in mediating costimulation have been well defined. The two key pathways involve (i) B7-1, B7-2 (members of the B7 family) and (ii) CD40, which are expressed on the APC, and their counter-receptors CD28 and CD40 ligand (CD40L) respectively expressed on T cells. A large body of evidence, both in vivo and in vitro, clearly defines the crucial roles played by B7-1, B7-2 and CD40 in providing T cell costimulation (26-36). Furthermore, the simultaneous blockade of signalling via CD28-B7 and CD40-CD40L in an allotransplant context prevented the onset of allograft rejection (37, 38). In vivo, targeting the B7/CD28 interaction has been shown to prevent T cell sensitisation to graft antigen, thereby prolonging graft survival (38, 39).

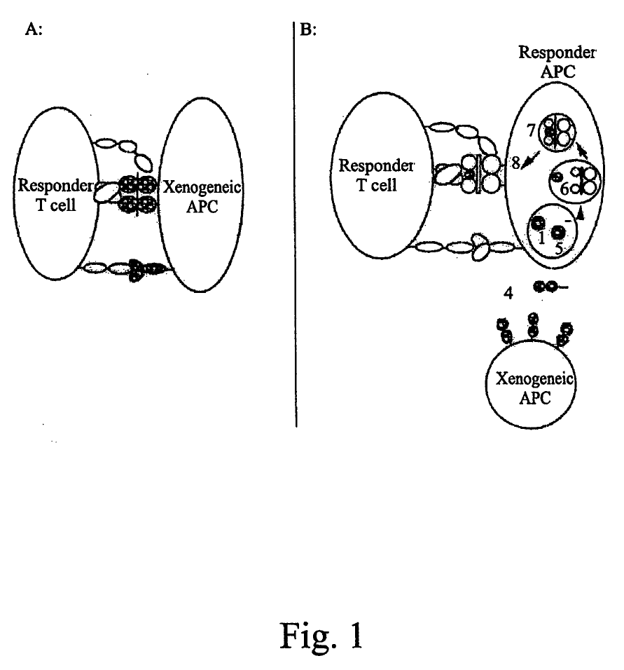

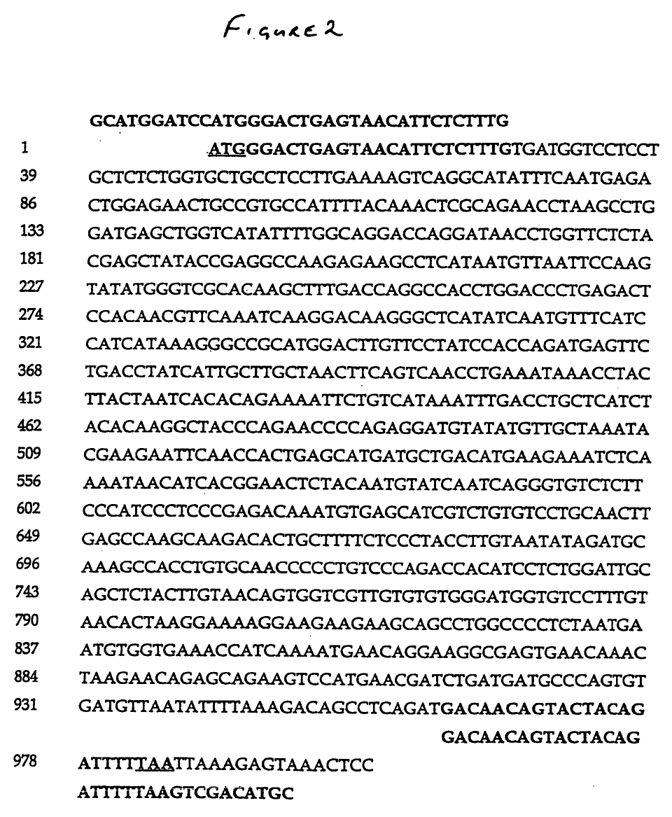

T cells can be sensitised against xenoantigens via one of two pathways—the direct and indirect pathways, which are analogous to the well documented T cell activation pathways against alloantigens (FIG. 1). Direct recognition requires that the recipient T cells recognise intact xeno MHC-molecules complexed with peptide on donor stimulator cells. In contrast, indirect recognition requires that recipient APC process the xenoantigen prior to presentation to recipient T cells in the context of recipient MHC II. Self MHC II restricted T cells with specificity for the xenoantigen will recognise the peptide and respond. Whilst the majority of data reported is of indirect xenorecognition responses, cell mediated rejection via the direct route has also been documented (7, 8, 9, 11, 12, 40, 41, 42). Vigorous human T cell proliferative responses directed against porcine tissues in vitro have been documented from studies both in this laboratory and others.

3.2 Costimulatory Molecules

The crucial role played by costimulatory molecules in determining the result of TCR-CD3 receptor engagement with MHC and peptides has been demonstrated extensively both in vivo and in vitro. Anti-costimulatory molecule strategies aimed at either the receptors or their ligands are being used as therapeutic strategies for altering the immune response. Such approaches have been tested in model transplant systems to alter cell mediated responses thereby preventing graft rejection (14, 37, 38, 43-47).

B7-1 (B7/BB1, CD80) and B7-2 (CD86) both belong to the immunoglobulin superfamily and are heavily glycosylated transmembrane proteins (25). B7-1, a B cell activation molecule was first identified in 1989 (27), followed by B7-2 in 1993 (49). Both human B7-1 and B7-2, and the murine homologues have now been cloned and functionally characterised (25). B7-1 and B7-2 are constitutively expressed on splenic and blood dendritic cells and are induced on B cells and monocytes upon activation (34, 50,). B7-1 and 2 are highly homologous and are the natural ligands for the T cell antigen CD28 (50). Cytotoxic T lymphocyte antigen-4 (CTLA4), a cell surface glycoprotein has been identified as a second receptor for the B7 family of molecules (51) and is homologous to CD28 with 31% sequence identity. Both B7 isoforms bind to CTLA4 with higher affinity than to CD28 (30, 50, 52). Whilst CD28-B7 receptor engagement results in an APC-derived costimulatory signal involved in antigen specific IL-2 production both in vivo and in vitro (53, 54), CTLA4 appears to function as a negative regulator of T cell activation (55, 56, 57). Cross-linking by anti-CTLA4 antibodies has been demonstrated to antagonise CD28 ligation (58) and, in addition, CTLA4 knock-out mice die due to uncontrolled lymphocyte proliferation within the first few weeks of life (59). Thus, CTLA4 ligation is thought to be crucial for the maintenance and regulation of immune responses. The underlying mechanisms have not, however, been clearly defined.

Among costimulatory molecules, the B7 family appears to be unique, since ligation by CD28 of either B7-1 or B7-2 is both necessary and sufficient to prevent the induction of anergy (34). The CD28-B7 interaction is thought to deliver crucial signals to sustain proliferation of activated T cells. These observations are supported by in vitro data showing that whilst cells deficient in B7 fail to stimulate a primary MLR, transfectants expressing high levels of B7 gained the capacity to stimulate the production of IL-2 by alloreactve T cells and to co-stimulate a polyclonal population of purified T cells cultured with immobilised anti-CD3 Mab (31). Artificial APC generated by stably transfecting NIH-3T3 cells with HLA-DR7, B7 or both, clearly demonstrated that following presentation of tetanus toxoid (TT) optimal T cell proliferation and IL-2 production resulted only when both molecules were present. In the absence of B7, clonal anergy resulted (58).

Porcine B7-2 (PoB7-2) has been cloned from aortic endothelial cells (60). Following transient transfection of porcine B7-2, human umbilical vein endothelial cells strongly costimulated IL-2 production by human T cells. This costimulation of human T cells by poB7-2 was shown to be as effective as costimulatory signals provided by human B7-1 or B7-2 and could be specifically blocked by huCTLA4Ig. Thus poB7-2 strongly contributes to the immunogenicity of porcine endothelium (60).

Although B7-1 and B7-2 mediated interactions appear to be central to the development of T cell specific immunity, additional costimulatory pathways of importance exist. The most crucial of which involves the CD40 and CD40 ligand (CD40L) interaction (34).

CD40 is a 50 kDa surface glycoprotein belonging to the TNF-receptor superfamily. CD40 is expressed on various APC including among others, monocytes, dendritic cells and activated macrophages. Other cell types including endothelium also express CD40 (34). Its counter-receptor CD40L (CD154, gp39, TRAP) is a 33 kDa type II integral membrane protein (34, 36) transiently expressed on activated CD4 T cells. The CD40-CD40L interaction has been demonstrated to play an important role in both the humoral and cellular arms of the immune response with a dominant role in B cell activation. Whilst cross linking of CD40 on B cells is essential for B cell growth and isotype switching, it also results in the upregulation of B7 expression (50). Levels of B7 expression (and thus APC capacity) of monocytes and dendritic cells are clearly unregulated following CD40 signalling (34). Data from CD40 knock-out mice demonstrated that CD40L signalling following ligation by CD40 plays an important role in T cell activation (61). Transfection of the murine P815 mastocytoma cells with CD40 (or B7-1) enabled previously non-stimulatory P815 cells to mediate the costimulation necessary for polyclonal T cell activation and the generation of cytokines (34). CD40-CD40L interactions have also been demonstrated to play a critical role in allograft rejection (62, 63).

Resting B cells do not normally express B7-1/B7-2 at high levels until they are activated (50). Activation of B cells following simultaneous engagement of MHC-peptide/TCR and CD40-CD40L leads to the upregulation of B7 family members on B cells, thereby enhancing the stimulation and subsequent activation of T cells (34, 36). Thus, the CD40-CD40L interaction influences costimulatory activity by inducing expression of the B7 family of molecules and perhaps other costimulatory molecules, thereby playing a key role in T cell activation. The clear synergistic effects of CD40 and B7 indicate the importance of both costimulatory pathways for the initiation and amplification of T cell dependent immune responses (38). CD40-CD40L interactions have also been shown to play a crucial role in the generation of cytotoxic T lymphocyte (CTL) responses by modifying the functional status of a dendritic cell (64, 65, 66)

Extensive studies have demonstrated the importance of blocking B7-CD28 and/or CD40-CD40L interactions in the context of both allo and xenotransplantation. Data strongly supporting this includes the use of CTLA4Ig to block signalling via CD28-B7 resulting in enhanced graft survival and the prevention of chronic rejection in a rat cardiac allograft model (44, 45) and a murine aortic allograft model (43). In these models, administration of CTLA4Ig caused partial (44) or complete (46) tolerance to graft antigen by inducing T cell anergy. Treatment of allo pancreatic islet transplants with anti-B7-2 and B7-1 antibody has also been demonstrated to inhibit transplant rejection (14). Similar results were obtained in models inhibiting CD40 signalling in a mouse cardiac allotransplant models (37, 47, 62). Two studies detailing the simultaneous blockade of signalling via CD28-B7 and CD40-CD40L prevented the onset of allorejection. Concurrent prolonged inhibition of both pathways completely abrogated the onset of chronic rejection in a mouse allo model (37) and in a skin and heart allo model (38).

In the realm of xenotransplantation, Lenshow and colleagues have, demonstrated long-term donor specific tolerance of human islets transplanted into mice with concomitant treatment with CTLA4Ig (46). Graft specific tolerance was demonstrated to be a direct consequence of inhibiting recognition via B7 expressing APC. In addition, Tran et al (67) demonstrated short term suppression with CTLA4-Fc treatment. There is limited data available on the simultaneous blockade of both pathways in the xenotransplantation context, with the prolonged survival of rat and porcine skin transplanted into murine recipients (63).

In vitro and in vivo data have clearly demonstrated that targeting the interactions mediated by either the CD28-B7, CD40-CD40L, or both pathways has prevented the sensitisation of T cells to alloantigen and xenoantigen from engrafted tissue thereby prolonging graft survival ( ).

As noted above, T-cell mediated graft rejection is well documented. The immune system can mount alternate or additional cell mediated rejection mechanisms. These mechanisms are illustrated by the function of various molecules expressed by, inter alia, endothelial cells. VCAM is a cell adhesion molecule, expressed by endothelial cells, that is thought to have a role in leukocyte recruitment to sites of inflammation. VCAM is an inducible transmembrane glycoprotein which has a basal level expression in resting endothelial cells but is rapidly expressed upon exposure to pro-inflammmatory cytokines (eg IL-1, TNFα). The interaction of VCAM with leukocytes is via the very late antigen 4 (VLA-4) expressed at the leukocyte cell surface. Therefore endothelial cell expression of VCAM functions to induce the infiltration of VLA-4 presenting leukocytes to sites of inflammation which augments rejection responses to allografts or xenografts.

It is believed that porcine VCAM plays an important role in allowing the migration of human leukocytes across porcine endothelial cell monolayers. There is a rationale for believing that blocking this interaction will have beneficial consequences on xenograft survival. Pig VCAM, cloned in 1994, has significant homology with human VCAM(1). As well as the data presented in (1), there is a wealth of evidence from other in vitro studies suggesting that pig VCAM interacts efficiently with human leukocyte-expression counter receptor, VLA-4. For instance, in static adhesion assays, antibodies to VCAM significantly inhibit the binding of human NK and T cells to pig endothelium. With NK cells, this disruption inhibits cell lysis which normally results after adhesion to porcine endothelial monolayers.

The effect of anti-VCAM antibodies on T cell mediated xenograft rejection mechanisms is more difficult to predict. In some rodent models of allotransplantation, antibodies against VCAM have been used to prolong allograft survival. In some instances, long term survival and specific tolerance have been described (2, 3), although the precise mechanism of action of these studies was not fully elucidated.

3.5 Peptide Immunisation Strategy

Previous in vivo studies using synthetic peptides conjugated to carrier molecules as immunogens have demonstrated the ability to generate the production of biologically active antibodies (68). There is now an extensive literature detailing peptide immunisation strategies which demonstrate enhancement of antibody production by carrier presentation (68-72). Thus, appropriate T cell epitopes can be used to prime T cells for subsequent help to B cells. Recent data has been published reporting the production of IgG by self-reactive B cells following immunisation with a self reacting antigen covalently coupled to a carrier molecule (70). Thereby demonstrating that B cell tolerance to self protein can be overcome.

As mentioned above, in order to be recognised by T cells, antigen (self or foreign) must be processed and presented by APC. B cells can act as highly potent APC following endocytosis of antigen via IgG receptors. In the presence of a full complement of activation signals (TCR engagement plus costimulation) T cell activation will occur resulting in the subsequent generation of antibody.

Peptides from self proteins are processed and presented to T cells in the same manner as foreign proteins, but because of T cell tolerance, presentation of self peptides does not normally result in T cell activation (70). The absence of T cell recognition may therefore explain, in part, why potentially reactive B cells fail to respond.

The ability to overcome B cell non-responsiveness to self peptides has recently been demonstrated by Dalum et al (69). An autoantibody response was generated by the provision of additional T cell help in the form of a strong foreign carrier T cell epitope. Further studies have demonstrated that synthetic peptides conjugated to T cell carrier molecules are capable of overcoming B cell non-responsiveness if significant numbers of self-reactive B cells are present in the host (69, 70). Insertion of a single foreign T cell epitope into the sequence of Ubiquitin, elicited strong autoantibody production directed against the native molecule (69). In an elegant study by Sad, using GnRH as a self protein chemically linked to diphtheria toxoid (DT) as the synthetic T cell epitope, autoantibodies were produced with specificity for native GnRH (71, 72). Following the initial vaccination, the continued presence of the native GnRH in vivo maintained the production of Ab. Continued antibody production caused sterility in the immunised mice due to the sustained anti-GnRH antibody response maintained by the continued presence of the native molecule against which the specific B cells were producing antibody. The DT carrier provoked a helper T cell response to assist GnRH specific B cells and break B cell tolerance.

### 4. STATEMENTS OF INVENTION

In its broadest aspect the invention relates to the immunisation of a mammal, preferably a human, with an immunogen which results in the production of antibodies specific to porcine epitopes expressed, typically, but not exclusively, by porcine endothelial cells which are involved in mediating xenograft tissue/organ immune rejection.

Immunogen is herein construed as any epitope or combination of epitopes capable of invoking an immune response. The epitope may be T cell specific or B-cell specific. In this context, epitope is construed as any polypeptide, peptide, modified polypeptide, modified peptide (eg typically modification may be by glycosylation or phosphorylation of the epitope).

Typically, the invention encompasses epitopes derived from porcine molecules which are selected from at least one of: CD40; B7.1; B7.2; VCAM.

It will be apparent to one skilled in the art that the invention provides means to immunise an individual, ideally prior to xenotransplantation, with an immunogen to a part of a porcine molecule which contains a B-cell epitope not present in the homologous mammalian polypeptide to ensure the selective production of antibodies to the porcine polypeptide without the development of antibodies to the patients own functional equivalent and without the development of CD4 T cell responses thereby avoiding cell mediated rejection. In addition the immunogen provides blocking antibodies generated by the recipient which abrogate the activity of porcine polypeptides which mediate a rejection response.

It will be still further apparent to one skilled in the art that the invention has significant advantages over prior art attempts to immunosupress a recipients immune system to porcine cells/tissues. For example, WO 97119971 discloses the use of B7.2 or VCAM polypeptides to produce diagnostic and therapeuctic antibodies to monitor transplantation rejection and to block xenotransplant rejection.

This has significant disadvantages. The treatment of a transplant patient with an antibody to, for example VCAM or B7.2, requires periodic administration throughout the life of the patient to maintain the blocking properties of the antibody. Moreover, the immune system will ultimately raise antibodies to the therapeutic antibodies (anti-idiotypic antibodies) resulting in their removal from the patients circulation.

The present invention does not require periodic administration since it is the patients own immune system that is responsible for the production of blocking antibodies to porcine polypeptides. The immune system will not recognise these antibodies as foreign and will therefore not result in the production of anti-idiotypic antibodies.

The present invention involves the use of a foreign T cell epitope to exert significant influences on subsequent responses to molecules conjugated to the carrier. By such means autoantibody responses may be directed against porcine polypeptides in a xenotransplantation context.

According to the present invention there is provided a method of improving the tolerance of an animal, including a human being, to a xenograft, the animal having T cell mediated immunity, the method comprising causing the animal to raise an antibody against a xeno-molecule involved in the generation of a rejection response in the animal, said antibody being raised by immunising the animal with a chimeric peptide comprising a T cell epitope against which the animal has immunity and a B cell epitope of said xenomolecule.

Accordingly, xenograft specific tolerance is induced in transplant recipients by targeting the direct T cell mediated response by the use of chimeric peptide constructs to stimulate the generation of specific anti-graft tolerance-promoting antibodies by the recipient prior to transplantation. By way of example, the chimeric peptides comprise a T cell epitope conjugated to sequences of porcine polypeptides, B7-1, B7-2, CD40, VCAM. The presence of the engrafted tissue will then serve to maintain and perpetuate the production of antibody by the recipient's B cells.

The present invention also provide a chimeric peptide comprising a T cell epitope and a B cell epitope, said T cell being that of an animal, including a human being of a first species and said B cell being of an animal of a second species, said first and second species such that xeno transplantations suitable from an animal of said second species to an animal of said first species.

In addition, the present invention provides the use of a chimeric peptide improving the tolerance of an animal, including a human being, to a xenograft, the chimeric peptide being as defined above.

According to a further aspect of the invention said immunogenic composition comprises at least one T-cell epitope and at least one B-cell epitope characterised in that said B-cell epitope is derived from at least one porcine polypeptide involved in mediating xenograft rejection and said T cell epitope is derived from a molecule to which the recipient is already immune.

In yet a further preferred embodiment of the invention said immunogenic composition comprises at least one peptide antigen derived from at least one of porcine: CD40; VCAM; CD86; CD80.

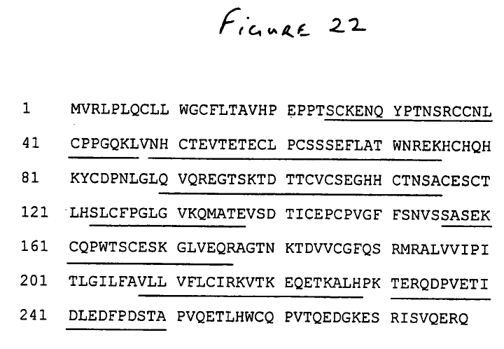

Preferably said peptide antigen is derived from porcine CD40. Ideally said peptide is derived from the amino-terminal domain of porcine CD40, or at least that part of the amino terminal domain that is exposed at the cell surface of a porcine cell presenting CD40. More ideally still said peptide antigen is selected from the peptide sequences presented in FIG. 22

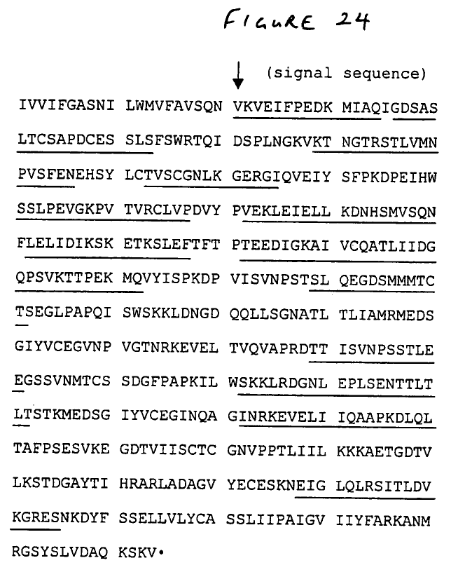

Preferably said peptide antigen is derived from porcine VCAM. Ideally said peptide is derived from the amino-terminal domain of porcine VCAM, or at least that part of the amino terminal domain that is exposed at the cell surface of a porcine cell presenting VCAM. More ideally still said peptide antigen is selected from the peptide sequences presented in FIG. 24

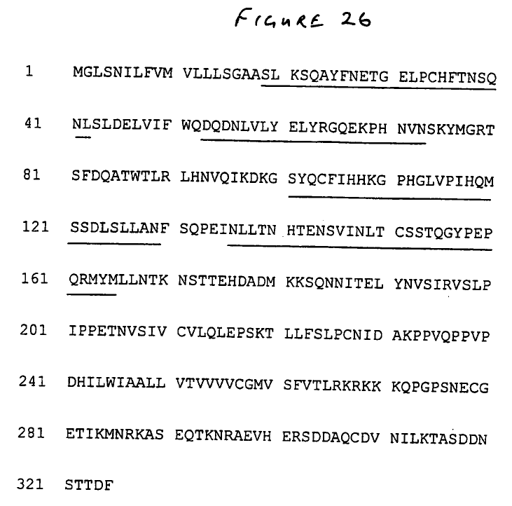

Preferably said peptide antigen is derived from porcine CD86. Ideally said peptide is derived from the amino-terminal domain of porcine CD86, or at least that part of the amino terminal domain that is exposed at the cell surface of a porcine cell presenting CD86. More ideally still said peptide antigen is selected from the peptide sequences presented in FIG. 26.

Preferably, said peptide antigen comprises at least 9 amino acid residues. More ideally still said peptide comprises 10-30 amino acid residues.

According to a further aspect of the invention there is provided an immunogenic composition according to any previous aspect or embodiment of the invention wherein said composition further comprises at least one agent capable of enhancing the immune response to said immunogenic composition.

In a preferred embodiment of the invention said agent is a carrier/adjuvant.

It is well known in the art that carriers/adjuvants are useful in promoting immune responses to selected antigens. These adjuvants are either crosslinked or coupled to the antigen or co-administered to the animal with the antigen. Adjuvants useful in promoting immune responses are detailed in Vaccine Design: The Subunit and Adjuvant Approach Chapter 7, p 141-228, Plenum Press, New York, 1995. Various carriers, excipients or diluants are available in which said immunogenic composition can be stored and/or administered. For example, and not by way of limitation, the encapsulation of the immunogenic composition in liposomes is a conventional practice. Liposomes are phospholipid based vesicles which are useful as carrying agents for immunogenic compositions and the like.

According to yet a further aspect of the invention there is provided an antibody, or at least the effective part thereof, directed to at least one region of at least one porcine polypeptide according to the invention.

In a preferred embodiment of the invention said antibody is a monoclonal antibody, or at least the effective part thereof. Ideally said antibody is labelled.

It will be apparent to one skilled in the art that antibodies according to the invention will have utility with respect to monitoring the expression of porcine polypeptides presented by porcine tissues/organs.

According to a further aspect of the invention there is provided a method to monitor the immune status of a mammalian recipient of a xenograft. Preferably said monitoring method is in vitro.

According to yet a further aspect of the invention there is provided a method to improve the tolerance of an animal to a xenograft comprising:
i) administering at least one immunogenic composition according to any previous aspect or embodiment of the invention to an animal; optionally
ii) monitoring the immune status of said animal to said immunogenic composition;
iii) transplantation of at least one porcine tissue/organ into said animal; and, optionally
iv) monitoring the animal for a rejection response to said porcine tissue/organ.

In a preferred method of the invention said animal is human.

In a further preferred method of the invention said xenograft is any vascularised graft and/or immunogenic porcine cell/tissue.

In a further preferred method of the invention said xenograft is porcine pancreatic islets.

It will be apparent to one skilled in the art that (ii) above can be conducted either by monitoring for the presence of antibodies to co-stimulatory molecules in sera (for example by ELISA or by FACS analysis of cells expressing said co-stimulatory molecules), or alternatively, or in addition, monitoring the presence of cytolytic T-cells in the blood of the treated animal by conventional T-cells lysis assays.

The potential benefits of the use of a chimeric peptide of the invention are that it avoids the need for injection of blocking antibodies or fusion proteins. Furthermore, the induction of a recipient antibody response circumvents the problems most commonly associated with administration of xenogeneic antibodies or fusions proteins, namely the immune response against the administered reagent.

An embodiment of the invention will now be described, by example only and with reference to the following Tables and Figures;

Table 1 represents the regions of non-homology in human CD40 with respect to the homologous porcine CD40;

Table 2 represents the regions of non-homology in human VCAM with respect to the homologous porcine VCAM;

Table 3 represents the regions of non-homology in human CD86 with respect to the homologous porcine CD86;

FIG. 1a is a diagrammatic representation of direct xenorecognition and FIG. 1b is a diagrammatic representation of indirect xenorecognition;

FIG. 2 represents the porcine CD86 nucleic acid sequence;

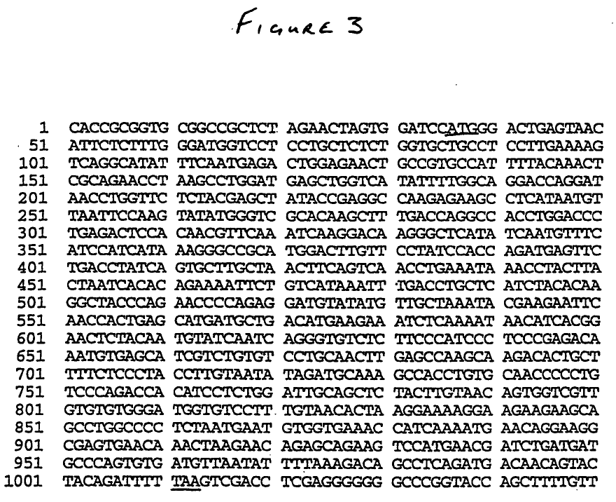

FIG. 3 represents the porcine CD86 cDNA sequence obtained by reverse transcription of porcine mRNA followed by PCR amplification;

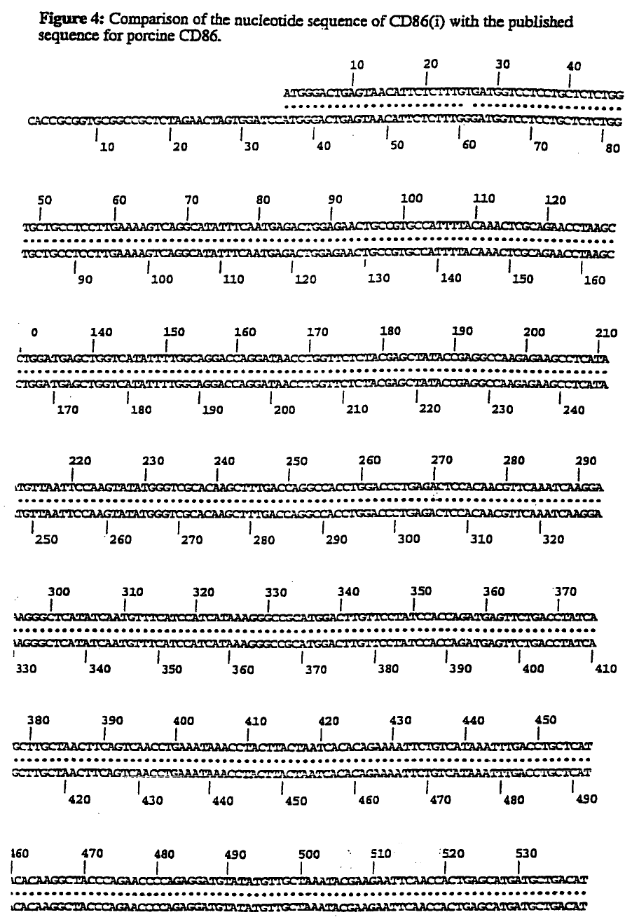

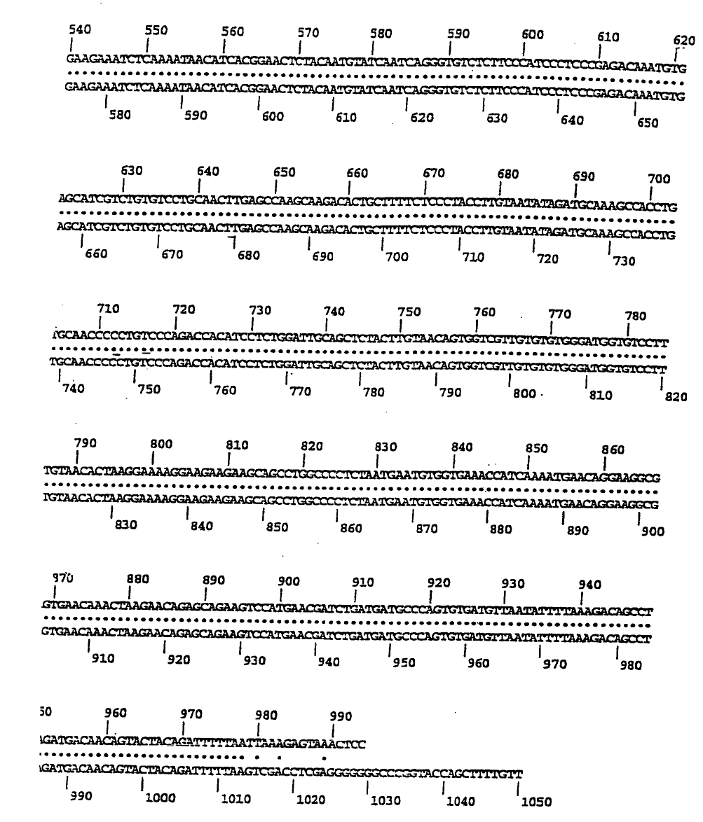

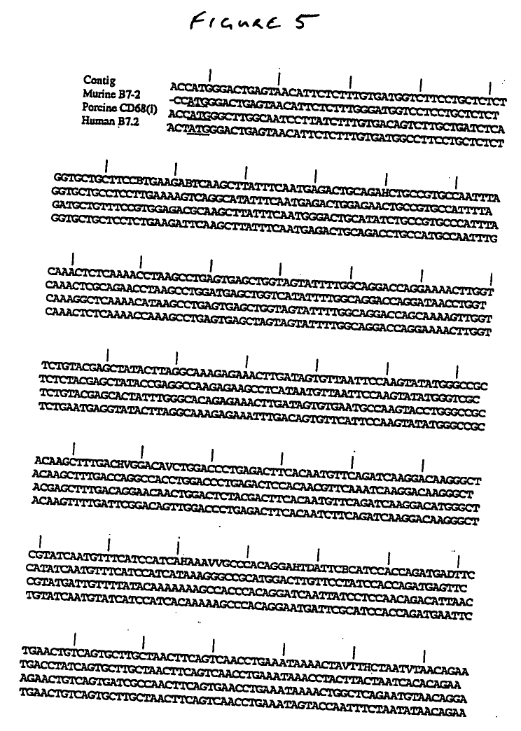

FIG. 4 represents a comparison of the nucleotide sequence of the cDNA in FIG. 2 with the published porcine CD86 sequence;

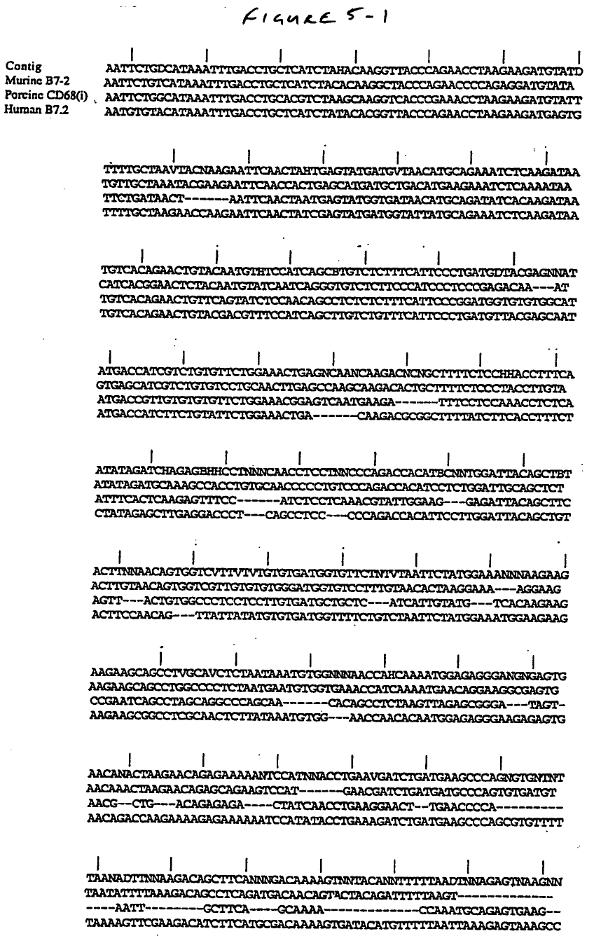

FIG. 5 represents a comparison of the cDNA sequence in FIG. 2 with the published murine and human CD86 sequences;

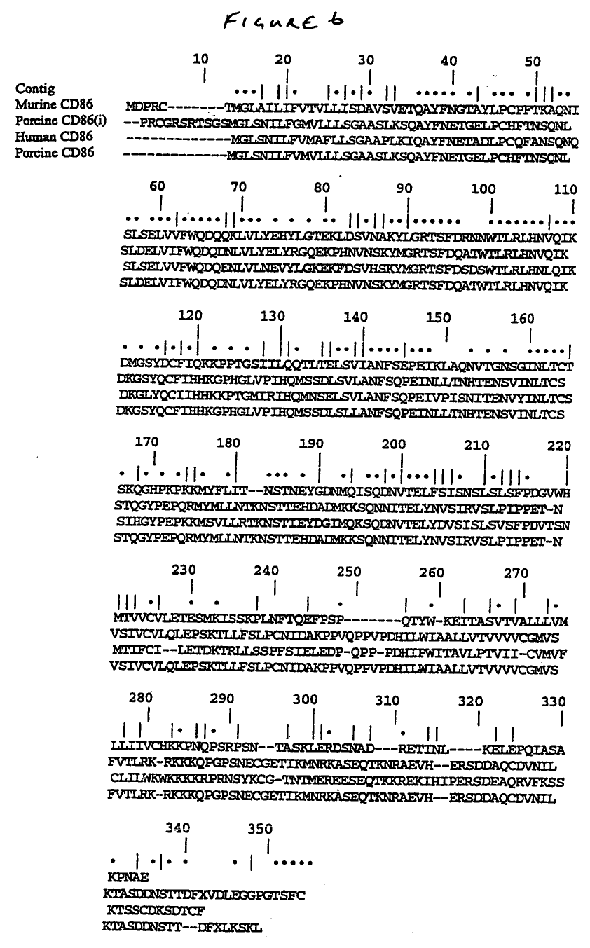

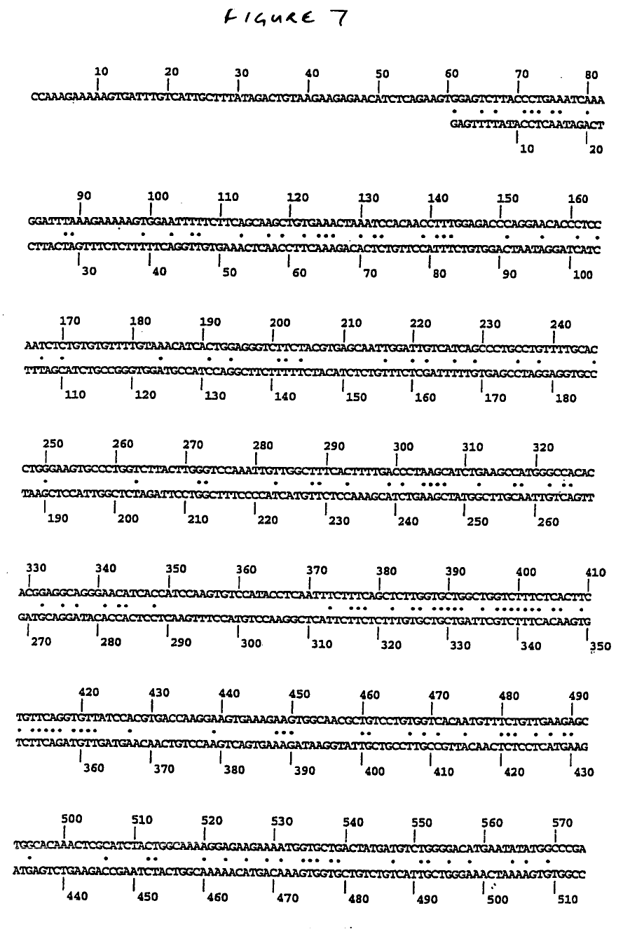

FIG. 6 represents the translated amino acid sequence of the cDNA in FIG. 2 compared with porcine, human and murine amino acid sequences;

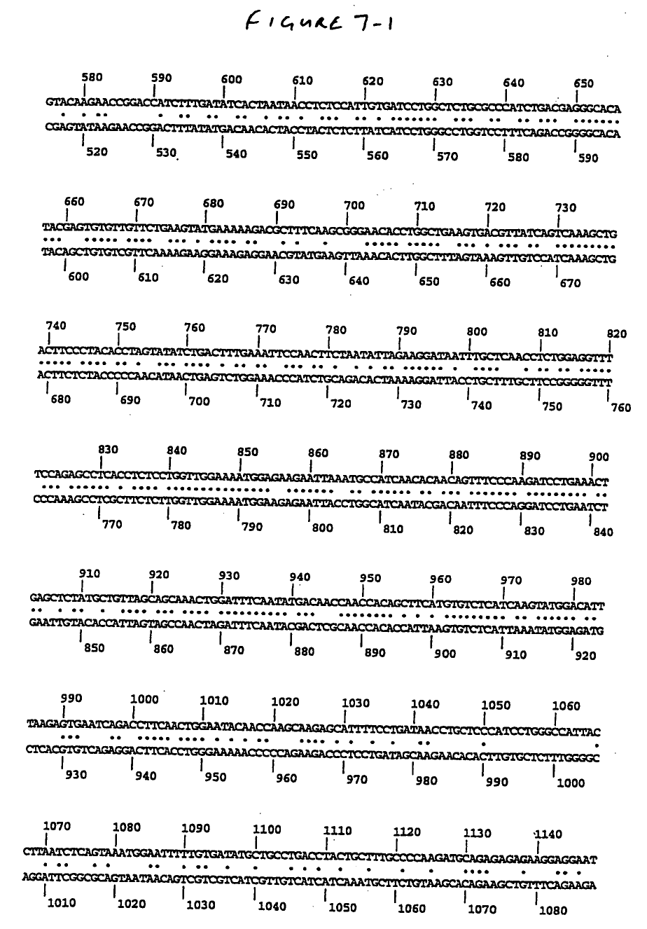

FIG. 7 represents the position of porcine B7.1 oligonucleotide primers with respect to the human and murine B7.1 nucleic acid sequences;

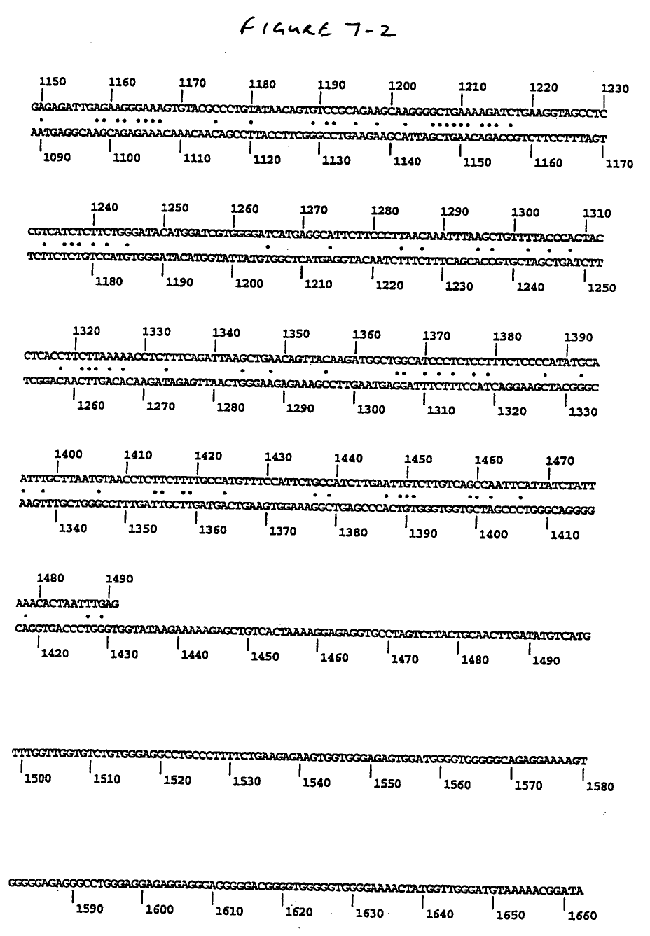

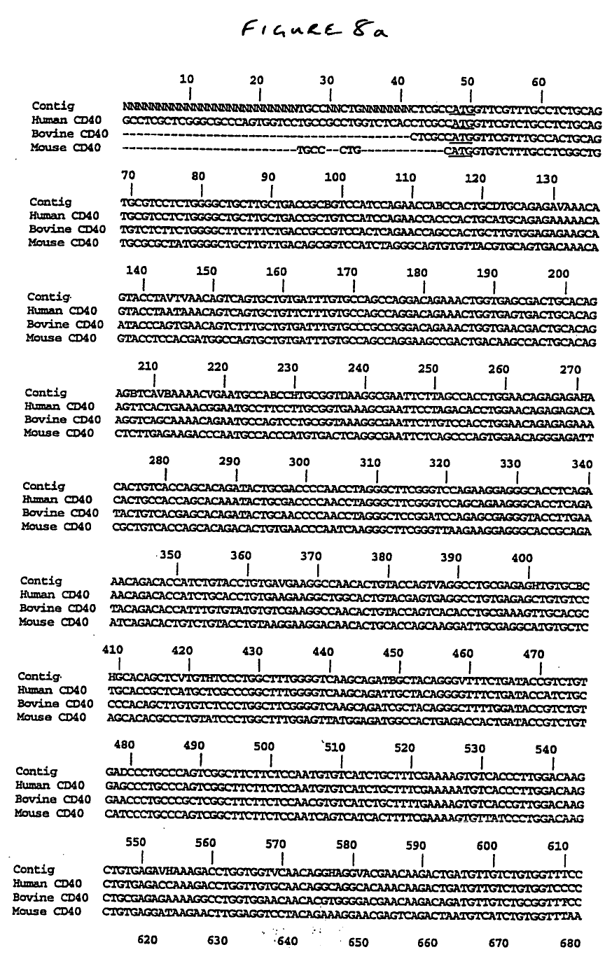

FIG. 8a represents a comparison of the human, murine and bovine CD40 nucleic acid sequences; FIG. 8b represents a comparison of the human, murine and bovine CD40 amino acid sequences;

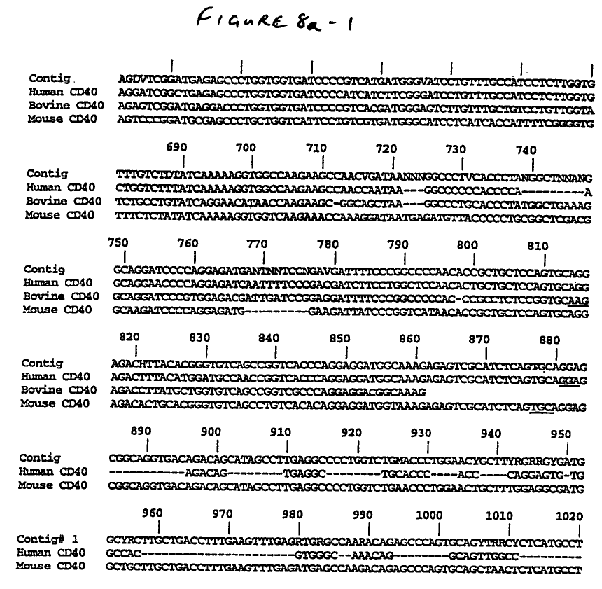

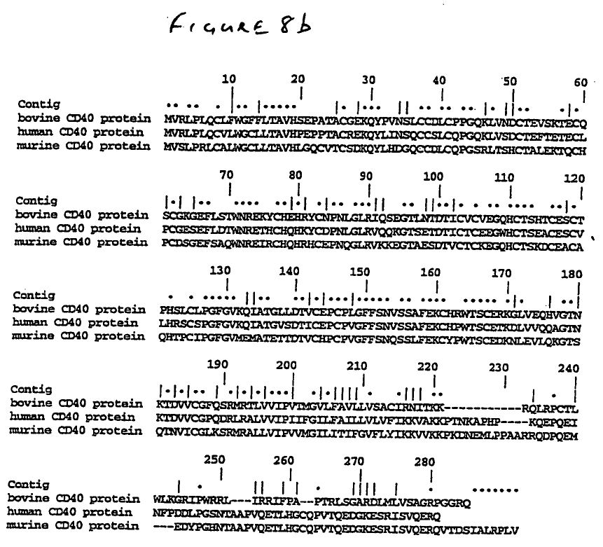

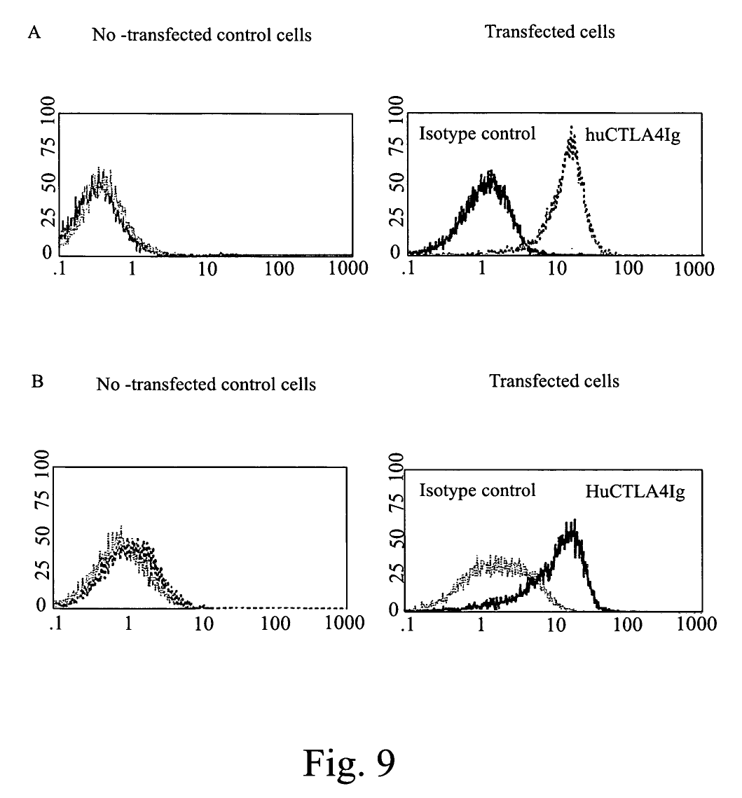

FIG. 9 represents FACS analysis of the expression of CD86 (B7.2) after transfection with a vector encoding porcine CD86 (B7.2);

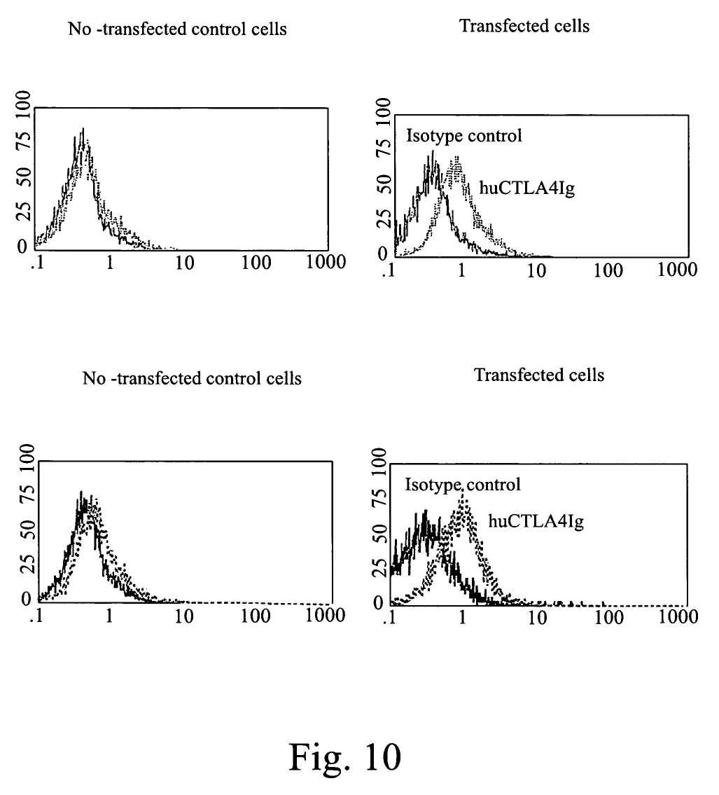

FIG. 10 represents FACS analysis of the expression of CD86 (B7.2) by transiently transfected cells with a vector encoding porcine CD86(B7.2);

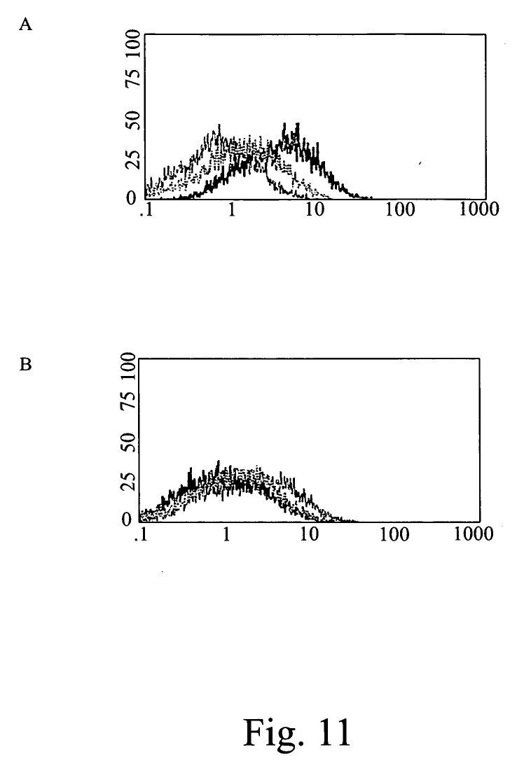

FIG. 11 represents flow cytometric analysis of cells transfected with porcine CD86(B7.2);

FIG. 12 represents the position of nine CD86(B7.2) derived peptides in the porcine CD86(B7.2) sequence;

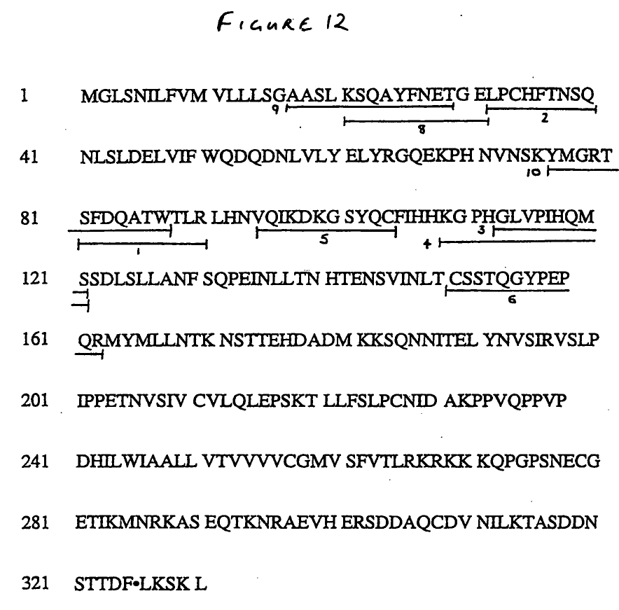

FIG. 13 represents a comparison of T cell proliferation response to whole ovalbumen or the ovalbumen peptide Ova₃₂₃₋₃₃₉;

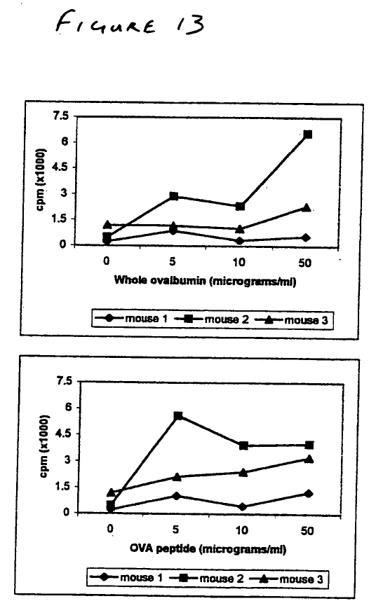

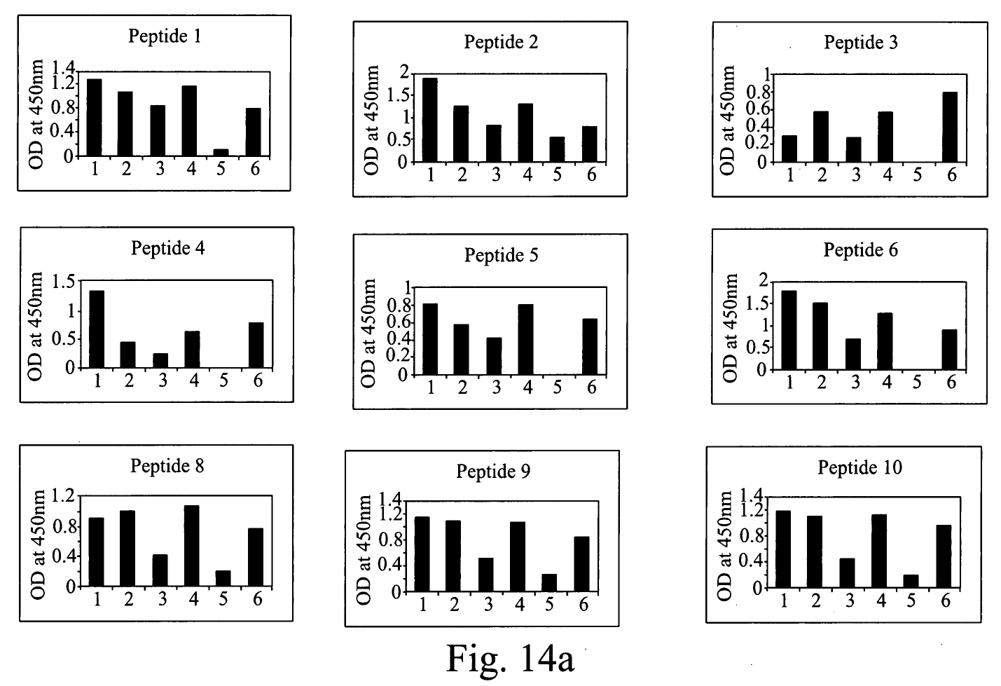

FIG. 14a represents the differential binding of B7.2 specific peptide sera or ovalburnen control sera by peptide ELISA;

FIG. 14b represents the in vitro recognition of B7.2 derived peptides 4 and 6 by mouse sera immunised with peptides 4 or 6;

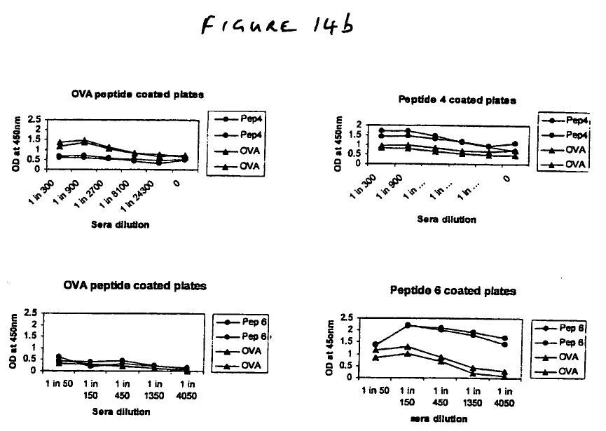

FIG. 15a represents the in vitro recognition of the B7.2 peptide sera and control ova peptide sera by peptide ELISA;

FIG. 15b represents the inhibition of direct mouse anti porcine T cell responses by peptide 4 and 6 sera which also shows no inhibition of of costimulation by murine CD86;

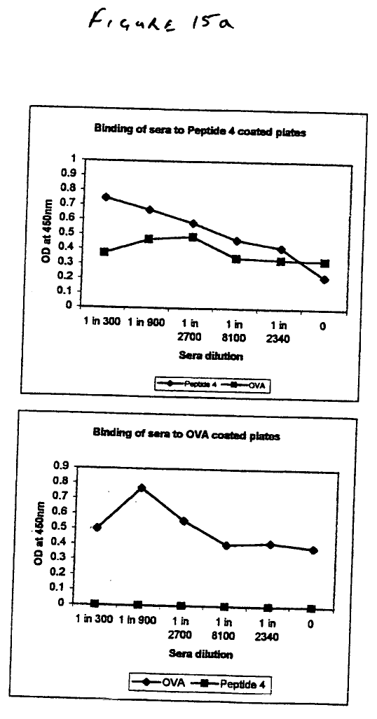

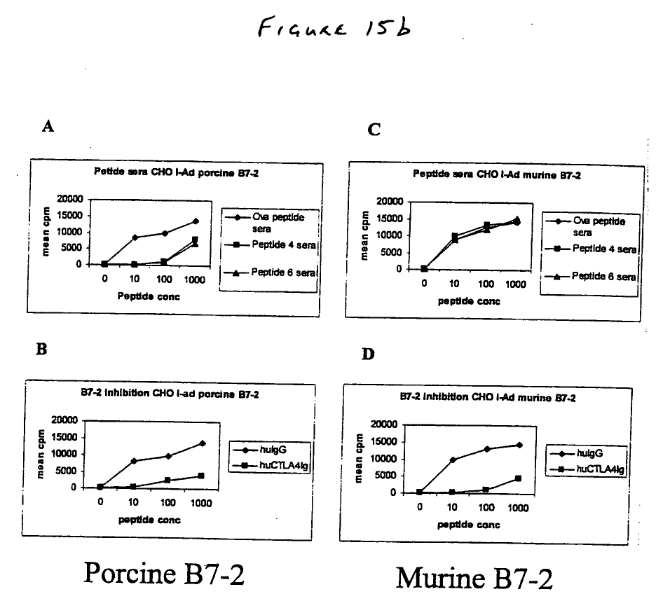

FIG. 16 represents the differential binding of the B7.2 derived peptide 4 sera or ova control peptide sera by peptide ELISA;

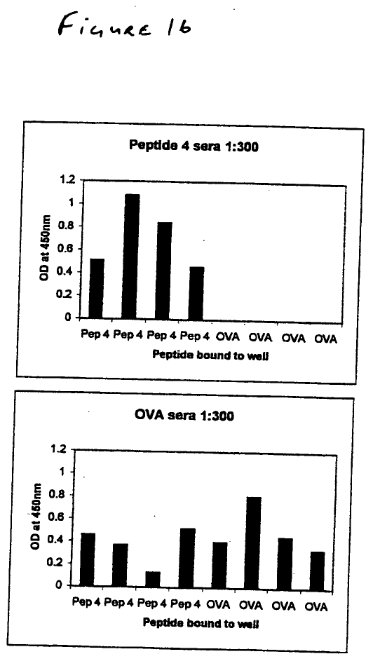

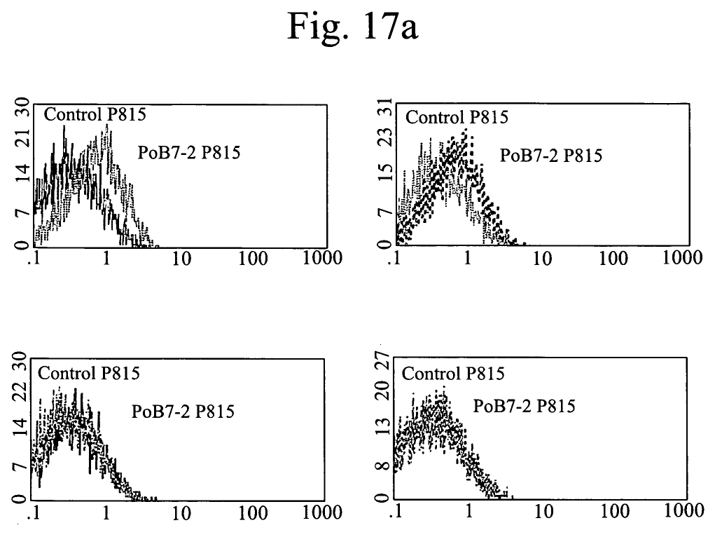

FIG. 17a represents flow cytometric analysis of P815 cells transfected with porcine CD86 following staining with sera from peptide 4 or control ova peptide sera;

FIG. 17b represents FACS analysis of P815 cells transfected with porcine CD86 or CHO cells transfected with murine CD86 following staining with sera from mice sera derived from peptide 4 or peptide 6;

FIG. 18 represents a preparation of porcine pancreatic islets isolated from a large white pig;

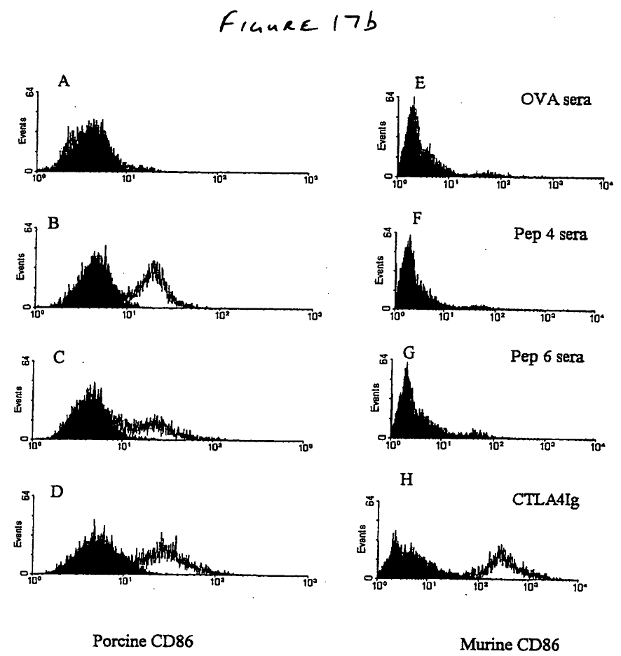

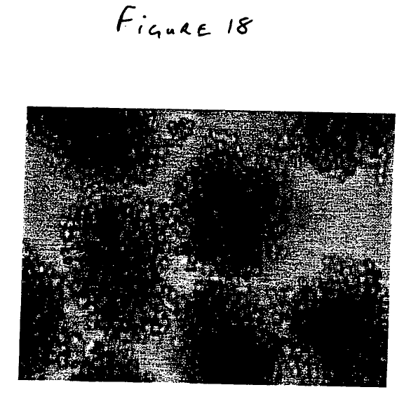

FIG. 19 is a schematic representation of the chimeric peptide immunisation and transplantation protocol;

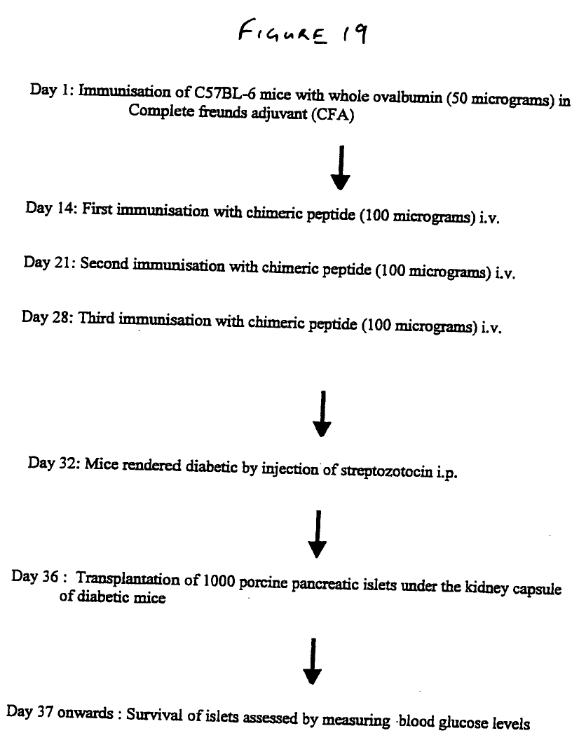

FIG. 20 shows that anti-porcine CD86 antisera prolongs the survival of transplanted porcine pancreatic islets;

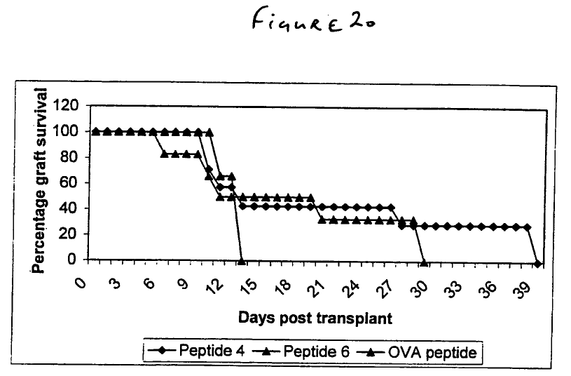

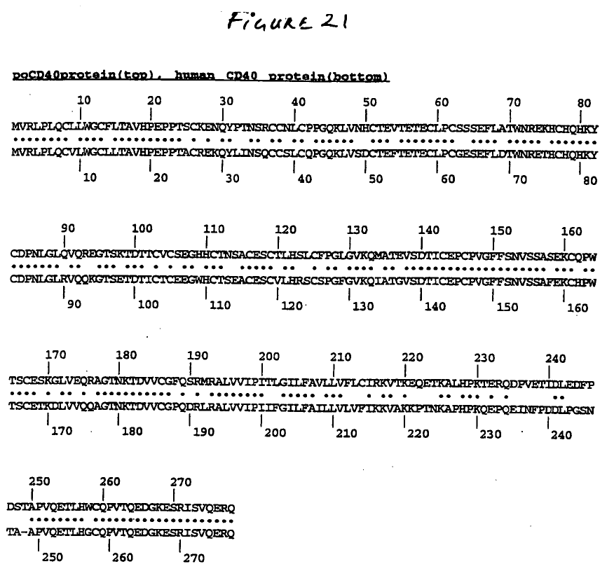

FIG. 21 is a comparison of the amino acid sequence of porcine and human CD40 (underlined sequences are peptides identified in table 1);

FIG. 22 is the translated amino acid sequence of porcine CD40 (underlined sequences are peptides identified in table 1);

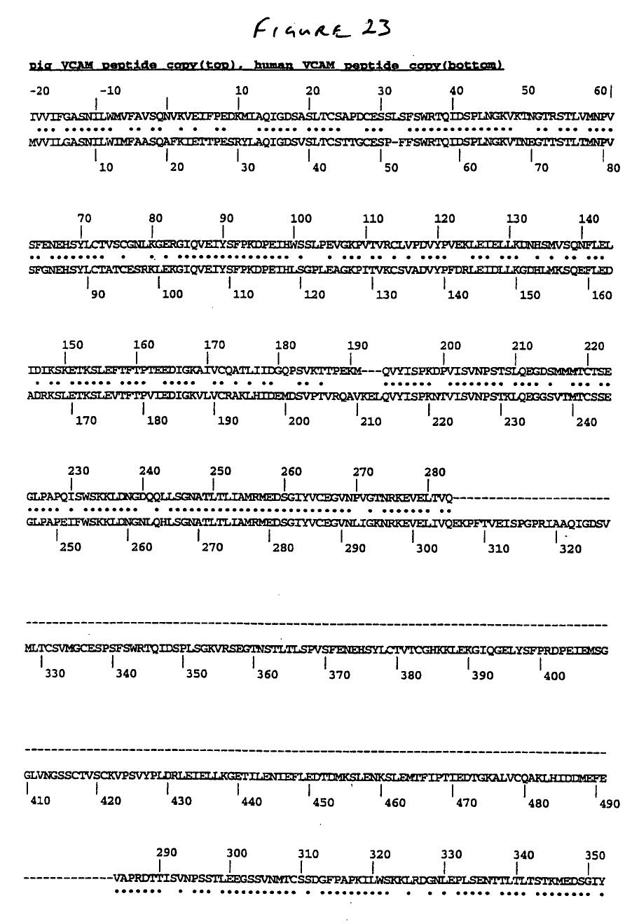

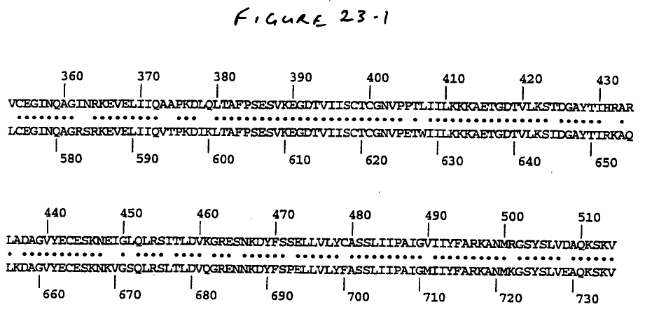

FIG. 23 is a comparison of the amino acid sequence of porcine and human VCAM (underlined sequences are peptides identified in table 2);

FIG. 24 is the translated amino acid sequence of porcine VCAM (underlined sequences are peptides identified in table 2);

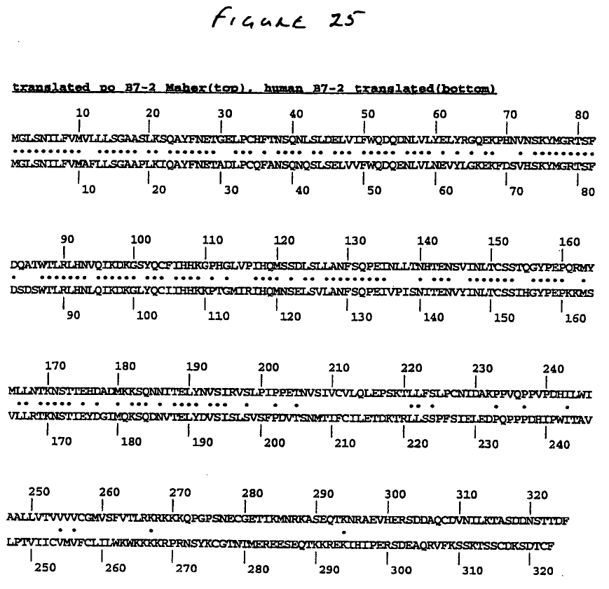

FIG. 25 is a comparison of the amino acid sequence of porcine and human CD86 (underlined sequences are peptides identified in table 3); and

FIG. 26 is the translated amino acid sequence of human CD86 (underlined sequences are peptides identified in table 3)

### 5. SPECIFIC EMBODIMENTS

5.1 Cloning Porcine Costimulatory Molecules

5.1.1 Cloning Porcine B7-2

RNA was extracted from primary and transformed porcine cells using a standard protocol. mRNA was then reverse transcribed and porcine B7-2 (poB7-2) amplified from the cDNA by 35 cycles of PCR at 56° C. with 1.5 mM magnesium. The 5′ and 3′ primers GCATGGATCCATGGGACTGAGTAACATTCTCTTTG and GCATGTCGACTTAAAAATCTGTAGTACTGTTGTC respectively were designed on the basis of the published poB7-2 sequence (60) to overlay the start and stop codons (FIG. 2). A 956 base pair fragment was generated and subcloned into the BamHI & Sal1 restriction sites of pbluescript. The nucleotide sequence was determined using standard m13 forward and reverse primers. The sequence of a single clone, CD86(i) is illustrated in FIG. 3, with comparison to the published sequences from porcine (FIG. 4), human and murine B7-2 (FIG. 5). One base pair difference is detected between our clone, CD86(i), and the published sequence at the 3′ prime end. This, however, is unlikely to be an important difference with respect to either poB7-2 expression or binding to its ligand. The predicted amino acid sequence of CD86(i), compared to that of porcine, human and mouse B7-2 is shown in FIG. 6.

5.1.2 Cloning Porcine B7-1 and CD40

RNA extracted from phytohaemagglutinin (PHA) or poke-weed mitogen (PMW) stimulated porcine PBMC and transformed porcine endothelial cells is being used to amplify cDNA encoding the costimulatory molecules B7-1 and CD40. B7-1 Primers were designed on the basis of conserved areas following comparison of murine and human (29, 49) sequences. External (lying outside the coding region) AGACCGTCTTCCTTTAG (3′i), TTGGATCCTCCATGTTATCCC (3′ii) and AGCATCTGAAGC (5′) and internal (within the coding region) ATGGATCCTCCATTTTCCAACC (3′) and TTGTCGACATCTACTGGC (5′) primers have been designed as depicted in FIG. 7. The generation of two 3′ primers is due to significant differences between the human and murine sequences in the terminal coding regions. Resulting PCR fragments will be subcloned as described above using the restriction sites BamHI and SalI contained within the promoter sequence. Constructs will then be sent for sequence confirmation.

CD40 primers were designed in a similar manner following sequence alignment of published CD40 sequences from human, mice and cattle (73, 74, 75) as illustrated in FIGS. 8A & B. The 5′ and 3′ primer sequences are GGATCCTCACTGTCTCTCCTGCACTGAGATGCGACTCTCCTCTTTGCCGTCCG TCCTCC and GAATTCATGGTTCTGTTGCCTCTGCAGTG respectively containing the BamHI and EcoRI restriction sites.

5.2 Generation of Porcine Costimulatory Molecule Expressing Cell Transfectants

The poB7-2 molecule (CD869(i)) has been subcloned into the eukaryotic expression vector pci.neo carrying the neomycin drug-selectable marker. This is being used to transfect M1 and M1.DR1 transformed murine cell lines using a standard calcium phosphate precipitation method. G418 resistant pci.neo expressing cells will be selected using dynabead purification and highly expressing clones is selected by limiting dilution.

Stable poB7-2 M1 and P815 transfectants have been generated by this approach using the poB7-2 DNA construct supplied to us by Maher et al (FIG. 9). transient transfections of M1 and P815 cells have been generated using our CD86(i) construct (FIG. 10). 3 particular assays are undertaken using the CD86(i) transfected cells.

(I) comparative costimulatory function of poB7-2 with human B7-1 in the context of MHC restriction;

(II) flow cytometric analysis of specific anti-poB7-2 antibodies in the sera of immunised mice; and

(III) generation of specific anti-poB7-2 monoclonal antibodies.

(I) Comparative in vitro analysis is performed to determine the costimulatory function of poB7-2 or poB7-1 in the context of the human MHC class II molecule HLA-DR1, with that of human B7-1 or B7-2 in the context of DR1, in proliferation assays with human or porcine responders.

(II) Transfected P815 cells are crucial reagents for the detection of porcine anti-B7-2 antibody in the sera of immunised mice which have undergone the chimeric peptide immunisation regimen. Flow cytometric analysis with control or poB7-2-transfected P815 cells, reflects the specificity of sera for B7-2. Preliminary studies with C57BL-6 mice immunised with a pool of all nine B7-2 peptides have demonstrated the preferential binding of B7-2 peptide sera to porcine B7-2 transfected P815 cells (FIG. 11a and 11b).

(III) Mab with specificity for poB7-2 are generated by immunisation of Balb/c mice with poB7-2 expressing P815 cells. The spleens from immunised mice are fused with the NS0 fusion partner and successful fusion's selected by virtue of HAT selection. Flow cytometric staining of poB7-2 P815 transfectants with culture supernatants enable the identification of MAb secreting cells. Cells are grown in culture and the medium harvested for antibody purification by passage over Protein G following ammonium sulphate precipitation. Techniques for the preparation on monoclonal antibodies are well known in the art and with reference to publications such as Harlow and Lane Antibodies; A Laboratory Manual; Cold Spring Harbour Laboratories.

MAb with specificity for B7-1 and CD40 are generated using the same protocol. These MAb will provide valuable reagents for further characterising the expression of CS molecules on relevant porcine tissues.

5.3 Design and Synthesis of poB7-2/OVA Chimeric Peptide Constructs

Nine different peptides derived from the sequence of poB7-2 were initially selected for synthesis. Porcine B7-2 peptides, 6-22mer in size, were selected as determined by the predicted size of a B cell epitope. Peptides were selected for synthesis in combination with a T cell epitope OVA 323-339. B7-2 peptides were selected on the basis of 3D computer modelling (in collaboration with Paul Travers) and on the basis of predicted antigenicity and hydrophilicity using the SeqAid II computer software package. All of the nine peptides reflect linear epitopes. The positions of the nine peptides in the cloned poB7-2 sequence are indicated (FIG. 12). Synthetic peptide sequences are detailed in Table 1

TABLE 1

| | | |
|---|---|---|
| Peptide Name | Peptide Sequence | Position |

| | | |
|---|---|---|
| Peptide 1 | ISQAVHAAHAEINEAGRSFDQATWTLR | 81-90 |
| Peptide 2 | ISQAVHAAHAEINEAGRLPCHFTNSQ | 32-40 |
| Peptide 3 | ISQAVHAAHAEINEAGRKGPHGLVPIHQMS | 109-121 |
| Peptide 4 | ISQAVHAAHAEINEAGRGLVPIHQMS | 113-121 |
| Peptide 5 | ISQAVHAAHAEINEAGRVQIKDKGSYQC | 94-104 |
| Peptide 6 | ISQAVHAAHAEINEAGRCSSTQGYPEPQR | 151-162 |
| Peptide 8 | ISQAVHAAHAEINEAGRKSQAYFNETGEL | 21-32 |
| Peptide 9 | ISQAVHAAHAEINEAGRASLKSQAYFNET | 17-29 |
| Peptide 10 | ISQAVHAAHAEINEAGRYMGRTSFDQATWT | 76-88 |
| Ova Peptide | ISQAVHAAHAEINEAGR | 323-339 |

The peptide sequences and amino acid positions for peptides 1-10 relate to the position of the B7-2 peptide sequence within porcine B7-2. The amino acid position for the ova sequence is only indicated for the Ova peptide. A 17 amino acid peptide from chicken egg albumin (ovalbumin) was selected as the T cell epitope, OVA323-339 (ISQAVHAAHAEINEAGR). This epitope was selected on the basis of published reports for the generation of a H-2^{b} restricted T cell response (76, 77). We have demonstrated the ability of C57BL-6 mice (H-2^{b} haplotype) to mount a proliferative response to both the native molecule and to the OVA 323-339 peptide following immunisation with whole ovalbumin (FIG. 13). Peptides were generated on a peptide synthesiser (Genosys) and crude peptides were purified by HPLC to greater than 70% purity. Sera from OVA control immunised mice should ideally not recognise the 323-339 sequence, indicating that the T cell epitope is devoid of B cell determinants.

5.4 Tolerance Induction

5.4.1 In Vivo Tolerance Induction Strategy

C57BL-6 mice are immunised with whole ovalbumin in CFA, followed by either control peptide (OVA peptide) or CS peptides (OVA-B7-2 constructs) for three weekly immunisations. Blood is collected following sacrifice and sera prepared using a standard technique. Presence of specific mouse anti-porcine B7-2 IgG and/or IgM Ab is detected by one of two strategies.

Peptide ELISAs are used to screen for the presence of anti-peptide antibody in the sera. Peptides are coated to plates by virtue of aldehyde linkages to allow free access of Ab to the peptide (78), Plates are coated with individual peptides or the ova control peptide to enable the identification of specific peptides of interest. To detect reactivity of sera with the native B7-2 molecule expressed on the surface of PoB7-2 transfected P815 cells, flow cytometry is performed following surface staining. Having identified CS peptide of interest (peptide ELISA positive and recognising native B7-2) the sera is used to inhibit in vitro T cell proliferative responses. This determines whether the antibody is a blocking antibody.

In vivo studies are performed using the islet transplant system. Antibodies which recognise the native molecule but fail to block a proliferative response are useful polyclonal antibody reagents.

Immunisations involved two groups of mice, one received a pool of all nine B7-2 peptides, and one receiving ova control peptide. The harvested sera were screened by peptide ELISA (FIG. 14a or 14b) which enabled the identification of peptides of interest. Antisera to peptides 2, 4 and 6 clearly demonstrate preferential binding to B7 peptide than to ova control. The sera has also demonstrated enhanced binding to poB7-2 transfected cells (FIG. 11). Peptide 4 and 6 were selected as candidate peptides and used in subsequent immunisation protocol. Immunisation with peptide 4 or 6 clearly produced a significant level of IgG with specificity for peptides 4 and 6 in the sera of immunised mice (FIGS. 15a and 15b). The specificity of the sera for peptide 4 and not to ova control is demonstrated in FIG. 16. The ability of sera from peptide 4 and 6 immunised mice to specifically recognise the native porcine B7-2 molecule expressed on the surface of porcine B7-2 transfected P815 cells is illustrated in FIGS. 17a and 17b. Untransfected control P815 cells do not stain with the Peptide 4 or 6 sera, neither do control or transfected cells incubated with ova peptide sera. Similar protocols will be followed with peptide 2. These data clearly demonstrate the ability of this technique to generate anti-peptide antibody directed against an amino acid sequence, by virtue of a carrier T cell epitope.

An identical strategy will be followed with peptides designed on the basis of porcine CD40 and porcine B7-1 once the DNA sequence encoding these molecules has been elucidated.

5.4.2 Functional Assessment; Prolongation of Pancreatic Islet Xenograft Survival

Islet xenografts being non-vascular are rejected solely by T cell mediated mechanisms (79, 80), thereby providing an ideal system to study modulation of T cell mediated reactions, please see FIG. 18. A very clear role for cell mediated rejection of islets has been demonstrated and is reported to be greater than the comparable alloresponse (80). Transplantation of porcine pancreatic islets to mice is an established procedure, which is well documented in the literature (80-83). Studies within this laboratory have demonstrated a decrease in hyperglycaemia (FIG. 18) following transplantation of pancreatic islets from large white pigs under the kidney capsule of C57BL-6 mice rendered diabetic by intraperitoneal administration of streptozotocin, please see FIGS. 19 and 20. Further optimisation of the isolation procedure (84, 85) is required to enable purification of fully functional islets. Transplanted islets usually survive between 6-10 days in the absence of any immunosupression. Successful modulation of direct T cell mediated xenorejection will be monitored by prolongation of islet survival beyond day 10, with comparison to the appropriate controls.

The results obtained with B7-2 to date, demonstrate the ability of synthetic B7-2 peptides conjugated to a known T cell helper epitope to generate the production of anti-pocine B7-2 antibody in vivo. These antibodies if directed towards the binding site between B7 isoforms and CD28, in association with antibodies directed against CD40-CD40L will block the costimulation of human T cells with direct anti-pig xenoreactivity thereby prolonging islet survival in a xenotransplantation context.

Having established the suitability of such an approach in a pig islet to mouse in vivo model, studies would progress to pig to primate transplantation systems prior to clinical trials.

5.5 Adaptations for Clinical Use of These Strategies

For clinical applicability the following requirements are necessary:

(I) selection of a suitable T cell epitope to replace OVA. One candidate molecule is tetanus toxiod (TT) which is a widely used antigen for use in human immunisation strategies (68, 86). The prior immunisations of most adults with TT is an additional benefit to this strategy as memory T cells are already present in the circulation.

(ii) An efficient and rapid screening method is used to detect the presence of anti-donor (pig) B7-2 antibodies in the absence of a specific B7-2 directed T cell response generated by the recipient which would accelerate graft rejection.

### 6. SUMMARY OF SPECIFIC EMBODIMENTS

The above examples relate to a novel strategy to inhibit costimulation by porcine cells of human T cells with direct anti-pig xenoreactivity. This is of particular importance in the context of xenotransplantation of porcine organs due to the expression of costimulatory molecules on porcine endothelial, as well as bone marrow-derived antigen presenting cells.

Recipients are immunised with hybrid synthetic peptides comprising a T cell epitope conjugated to sequences of the porcine costimulatory molecules, CD80, CD86 and CD40. Peptides that induce antibodies specific for regions of the costimulatory molecules involved in binding to their counter-receptors on human cells (CD28 and CD154) are therefore capable of blocking the delivery of costimulation. Once the antibody response has been induced, the transplanted organ will recall this response due to the expression of the costimulatory molecules, thereby sustaining this response, and providing an endogenous mechanism of costimulatory blockade.

### 7. BIBLIOGRAPHY

1. Dorling, A. et al. Clinical Xenotransplantation. Lancet. (1997). 349:867-71.
2. Cooper, D. K. C. Xenografting: how great is the clinical need. Xeno. (1995). 1: 25-26
3. Advisory Group on the Ethics of Xenotransplantation. Animal Tissue into Humans. London: Stationery Office, 1997.
4. Nuffield Council on Bioethics. Animal-to-human transplants. London: Nuffield Foundation, 1996.
5. van Denderen, B. J. et al. Combination of decay-accelerating factor expression and alpha 1,3-galactosyltransferase knockout affords added protection from human complement-mediated injury. Transplantation. (1997). 64.882-888.
6. Thompson, C. Humanised pigs hearts boost xenotransplantation. Lancet (1995): 346: 766.
7. Dorling, A. et al. Detection of primary direct and indirect human anti-porcine T cell responses using a porcine dendritic cell population. European Journal of Immunology (1996): 26: 1378.
8. Dorling, A. et al. Cellular xenoresponses: Observation of significant primary indirect human T cell anti-pig xenoresponses using co-stimulator-deficient or SLA class II-negative porcine stimulators. Xenotransplantation (1996): 3: 112.
9. Kirk, AD. et al. In-vitro analysis of the human anti-porcine T-cell repertoire. Transplantation Proceedings. (1992): 24: 602.
10. Murray, AG. et al. Porcine aortic endothelial cells activate human T cells: Direct presentation of MHC antigens and costimulation by ligands for human CD2 and CD28. Immunity (1994): 1: 57.
11. Yamada, K. et al. Human anti-porcine xenogeneic T cell response. The Journal of Immunology. (1995). 155: 5249-5256.
12. Kumagai-Braesch, M. et al. Characteristics of direct and indirect activation of human T cells against allogeneic and porcine xenogeneic cells/peptides. Xenotransplantation. (1997). 4: 85-94.
13. Dorling, A. and Lechler, R. I. The passenger leukocyte, dendritic cell and antigen-presenting cells (APC), In Transplantation Biology; Cellular and Molecular Aspects. Eds N. L. Tilney, T. B. Strom and L. C. Paul. Philadelphia: Lippincott-Raven, 1996.
14. Lenschow, D. J. et al. Inhibition of transplant rejection following treatment with anti-B7.1 antibodies. Transplantation. (1995). 60: 1171-1178.
15. Bretscher, P. and Cohen, M. A theory of self-nonself discrimination. Science (1970): 169: 1042.
16. Bretscher, P. The two signal theory of lymphocyte activation twenty one years later. Immunology Today. (1992). 13 : 74-76.
17. Mueller, D. L. et al. Clonal expansion versus functional clonal inactivation: A costimulatory pathway determines the outcome of T cell receptor occupancy. Annual Reviews of Immunology. (1989). 7: 445-480.
18. Mueller, D. L. et al. An accessory cell-derived costimulatory signal acts independently of protein kinase C activation to allow T cell proliferation and prevent the induction of unresponsiveness. The Journal of Immunology. 142: 2617-2628.
19. Baird, M. A. Evidence that heat-treated antigen-presenting cells induce hyporesponsiveness in allogeneic T cells. Transplantation. (1994): 57: 763.
20. Jenkins, M. K. et al. Molecular Events in the induction of a non-responsive state in interleukin 2 producing helper T-Lymphocyte clones. Proceedings of the National Academy of Science USA (1987): 84: 5409.
21. Inaba, K. and Steinman, R M. Resting and sensitized T lymphocytes exhibit distinct stimulatory (antigen-presenting cell) requirements for growth and lymphokine release. Journal of Experimental Medicine (1984): 160: 1717.
22. Schwartz, R. H. A cell culture model for T lymphocyte clonal anergy. Science. (1990). 248: 1349-1355.
23. Lafferty, K. J. et al. Immunobiology of tissue transplantation: A return to the passenger leukocyte concept. Annual Reviews of Immunology. (1983): 1: 143.
24. Jenkins, M. K. and Schwartz, R. H. Antigen presentation by chemically modified splenocytes induces antigen-specific T cell unresponsiveness in vivo and in vitro. The Journal of Experimental Medicine. (1986). 165: 302-319.
25. Schultze, J. et al. B7-mediated costimulation and the immune response. Blood Reviews. (1996). 10: 111-127.
26. June, C. H. et al. The B7 and CD28 receptor families. Immunology Today (1994): 15: 321.
27. Freeman, G. J. et al. B7, A new member of the Ig Superfamily with unique expression on activation and neoplastic B cells. Journal of Immunology. (1989): 143: 2714.
28. Freeman, G. J. et al. Cloning of B7-2: A CTLA-4 counter receptor that co-stimulates human T cell proliferation. Science (1993): 262: 909.
29. Azuma, M. et al. B70 antigen is a second ligand for CTLA-4 and CD28. Nature (1993): 366: 76.
30. Linsley, P. S. T-cell antigen CD28 mediates adhesion with B cells by interacting with activation antigen B7/BB1. Proceedings of the National Academy of Science USA (1990): 87: 5031.
31. Norton, S. D. et al. The CD28 Ligand B7, Enhances IL-2 Production by Providing a Costimulatory Signal to T Cells. Journal of Immunology (1992): 149: 1556.
32. Galvin, F. et al. Murine B7 antigen provides a sufficient costimulatory signal for antigen-specific and MHC-restricted T cell activation. Journal of Immunology (1992): 149: 3802.
33. Boussiotis, V A. et al. Activated human B lymphocytes express three CTLA4 counterreceptors that costimulate T-cell activation. Proceedings of the National Academy of Science. USA (1993): 90: 11059.
34. vanGool, S. W. CD80, CD86 and CD40 provide accessory signals in a multiple step T cell activation model. (1996). 153: 47-83.
35. Tang, A. et al. Blockade of CD40-CD40 ligand pathway induces tolerance in murine contact hypersensitivity. Eurpean Journal of Immunology. (1997). 27: 3143-3150.
36. Grewal, I. S. and Flavell, R. A. The role of CD40 ligand in costimulation and T cell activation. Immunological Reviews. (1996). 153: 86-106.
37. Sun, H. et al. Prevention of chronic rejection in mouse aortic allografts by combined treatment with CTLA4Ig and anti-CD40 ligand monoclonal antibody. Transplantation. (1997). 64: 1838-1856.
38. Larsen, C. P. et al. Longterm acceptance of skin and cardiac allografts after blocking CD40 and CD28 pathways. Nature. (1996). 381: 434-441.
39. Wecker, H. and Auchincloss, H. Cellular mechanisms of rejection. Current Opinion in Immunology. (1992). 4: 561-566.
40. Satake, M. et al. Direct activation of human responder T cells by porcine stimulator cells leads to T cell proliferation and cytotoxic T cell development. Xenotransplantation. (1996). 3: 198-206.
41. Kirk, A. D. et al. The human anti-porcine T cell repertoire. In vitro studies of acquired and innate cellular responsiveness. Transplantation. (1993). 55 : 924-931.
42. Alter, B. and Bach, F. H. Cellular basis of the proliferative response of human T cells to mouse xenoantigens. Journal of Experimental Medicine. (1990). 171: 333-338.
43. Baliga, P. et al. CTLA4Ig prolongs allograft survival while suppressing cell mediated immunity. Transplantation (1994): 58: 1082.
44. Turka, L A. T cell activation by the CD28 ligand B7 is required for cardiac allograft rejection in vivo. Proceedings of the National Academy of Science. USA (1992): 89: 11102.
45. Lin, H. et al Long term acceptance of major histocompatibility complex mismatched cardiac allograft induced by CTLA4-Ig plus donor specific transfusion. Journal of Experimental Medicine (1993). 178: 1801.
46. Lenschow, D J. et al. Long term survival of xenogeneic pancreatic islet grafts induced by CTLA4-Ig. Science. (1992): 257: 789.
47. Lu, L. et al. Blockade of the CD40-CD40 ligand pathway potentiates the capacity of donor derived dendritic cell progenitors to induce long-term cardiac allograft survival. Transplantation. (1997). 64: 1808-1815
48. Fallarino, F. et al. B7-1 engagement of cytotoxic T lymphocyte antigen 4 inhibits T cell activation in the absence of CD28. Journal of Experimental Medicine. (1988). 188 : 205-210.
49. Freeman, G. J. et al. Murine B7-2, an alternative CTLA4 counter-receptor that costimulates T cell proliferation and IL-2 production. Journal of Experimental Medicine. (1993). 178: 2185-2192.
50. Jenkins, K. M. and Johnson, J. G. Molecules involved in T-cell costimulation. Current Opinion in Immunology. (1993) 5 : 361-367.
51. Brunet, J. F. et al. A new member of the immunoglobulin superfamily—CTLA-4. Nature (1987). 328: 267.
52. Lenschow, D. J. et al. B7 system of T cell costimulation. Annual Reviews of Immunology. (1996). 14: 233-258.
53. Norton, S. D. The CD28 ligand, B7, enhanes IL-2 production by providing a costimulatory signal to T cells. The Journal of Immunology. (1992). 149: 1556-1561.
54. Linsley, P. S. et al. T cell antigen CD28 mediates adhesion with B cells by interacting with activation antigen B7/BB1. Proceedings of the National Academy of Science. (1990). 87: 5031-5035.
55. Krummel, M. F. et al. CD28 and CTLA-4 have opposing effects on the response of T cells to stimulation. Journal of Experimental Medicine (1995): 182: 459.
56. Krummel, M. F. and Allison, J. P. CTLA-4 engagement inhibits IL-2 accumulation and cell cycle progression upon activation of resting T cells. Journal of Experimental Medicine (1996): 183: 2533.
57. Walunas, T. L. et al. CTLA-4 ligation blocks CD28-dependent T cell activation. Journal of Experimental Medicine (1996). 183: 2541.
58. Gimmi, C. D. et al. Human T-cell clonal anergy is induced by antigen presentation in the absence of B7 costimulation. Proceedings of the National Academy Science. USA (1993): 90: 6586.
59. Waterhouse, P. et al. Lymphoproliferative disorders with early lethality in mice deficient in CTLA4. Science (1995): 270: 985.
60. Maher, S. E. et al. Porcine endothelial CD86 is a major costimulator of xenogeneic human T cells. The Journal of Immunology. (1996). 157: 3838-3844.
61. vanEssen, D. et al. CD40 ligand-transduced co-stimulation of T cells in the development of helper function. Nature. (1995) 378. 620-623.
62. Larsen, C. P. et al. CD40-gp39 interactions play a critical role during allograft rejection. Transplantation. (1996). 61: 4-9.
63. Larsen, C. P. and Pearson, T. C. The CD40 pathway in allograft rejection, acceptance and tolerance. Current Opinion in Immunology. (1997). 9: 641-647.
64. Bennet, S. R. M. et al. Help for cytotoxic-T-cell responses is mediated by CD40 signalling. Nature. (1998). 393: 478-480.
65. Schoenberger. S. P. et al. T cell help for cytotoxic T lymphocytes is mediated by CD40-CD40L interactions. Nature. (1998) 393: 480-483.
66. Ridge, J. P. et al. A conditioned dendritic cell can be a temporal bridge between a CD4 T helper and a T-killer cell. Nature. (1998) 393: 474-478.
67. Tran, H. M. et al. Short-term xeno-suppression of the xeno-immune response with mCTLA4-Fc treatment. Transplantation. (1997). 4: 222-227
68. Lise, L. D. et al. Enhanced epitopic responses to a synthetic human malarial peptide by preimmunisation with tetanus toxoid carrier. Infection and Immunity. (1987). 55: 2658-2661.
69. Dalum, I. et al. Breaking of B cell tolerance toward a highly conserved self protein. The Journal of Immunology. (1996). 157: 4796-4804.
70. Dalum, I. et al. Induction of cross-reactive antibodies against a self-protein by immunisation with a modified self protein containing a foreign T helper epitope. Molecular Immunology. (1997). 34: 1113-1120.
71. Sad, S. et al. Bypass of carrier induced epitope-specific suppression using a T helper epitope. Immunology. (1992). 76: 599-603.
72. Sad, S. et al. Carrier induced suppression of the antibody response to a “self”-hapten. Immunology. (1991). 74: 223-227.
73. Grimaldi, J. C. et al. Genomic structure and chromosomal mapping of the murine CD40 gene. The Journal of Immunology. (1992). 149: 3921-3926.
74. Stamenkovic, I. et al. A B lymphocyte activation molecule related to the nerve growth receptor and induced by cytokines in carcinomas. The EMBO Journal. (1989). 8: 1403-1410.
75. Ramesh, N. et al. Chromosomal localisation of the gene for human B-cell antigen CD40. Somatic Cell and Molecular Genetics. (1993). 19: 295-298.
76. Shimonkevitz, R. et al. Antigen recognition by H-2-restricted T cells. The Journal of Immunology. (1984). 133: 2067-2074.
77. Robinson, J. H. et al. Palmitic acid conjugation of a protein antigen enhances major histocompatibility complex class II restricted presentation to T cells. Immunology. (1992) 76 : 593-598.
78. Elma, E. M. G. et al. Direct coating of poly(lys) or acetyl-thio-acetyl peptides to polystyrene: The effects in an enzyme-linked immunosorbent asay. Analytical Biochemistry. (1997). 248: 117-129.
79. Wennberg, L. et al. Allogeneic and xenogeneic islets are rejected by different and specific mechanisms: A study in rodents using a mixed allogeneic-xenogeneic islet transplantation model. Xenotransplantation. (1997). 4: 228-234.
80. Mandel, T. E. et al. Cellular rejection of fetal pancreas grafts: differences between allo and xenograft rejection. Xenotransplantation. (1997) 4: 2-10.
81. Mandel, T. E. et al. Transplantation of organ cultured fetal pig pancreas in non-obese diabetic (NOD) mice and primates (Macaca fascicularis). Xenotransplantation. (1995) 2: 128-132.
82. Lu, X. et al. Long-term survival of hamster islet xenografts in mice under short course treatment with non depleting versus depleting anti-CD4 monoclonal antibodies. Xenotransplantation. (1998). 5: 154-163.
83. Marchetti, P. et al. Automated large-scale isolation, in vitro function and xenotransplantation of porcine islets of langerhans. Transplantation. (1991). 52: 209-213.
84. Ricordi, C. et al. A method for the mass isolation of islets from the adult pig pancreas. Diabetes. (1986) 35: 649-653.
85. Ricordi, C. et al. Isolation of the eulsive pig islet. Sugery. (1990). 107: 688-694.
86. Tsang et al. Cloning and expression kinetics of porcine vascular cell adhesion molecule. BBRC (1994): 201: 805.
87. Orosz et al Treatment with anti vascular cell adhesion molecule-1 monoclonal antibody induces long-term murine cardiac allograft acceptance. Transplantation (1993): 53: 453.
88. Isobe et al. Immunosupression to cardiac allografts and soluble antigens by anti-vascular cellular adhesion molecule-1 and anti-very late antigen-monoclonal antibodies. J. Immunology (1994) 153: 5810.
86. Etlinger, H. M. et al. Use of prior vaccinations for the development of new vaccines. Science. (1990). 249: 423-425.
   CD86 (B7-2)

Human and porcine CD86 protein sequences were aligned and regions of non-homology identified. We predict that the peptide sequences will be derived from those regions listed below or from any overlap regions between any of these peptides.

The sequences of predicted interest for containing potential antibody epitopes have been selected on the basis of less than 75% sequence identity.

| Region | Position | % sequence identity |
|---|---|---|
| i | 18-42 | 72% |
| ii | 55-73 | 55% |
| iii | 101-127 | 63% |
| iv | 136-165 | 56% |

Regions (iii) and (iv) encompass those containing the peptide 4 and 6 sequences identitifed in mice.

CD40

Human and porcine CD40 protein sequences were aligned and regions of non-homology identified. We predict that the peptide sequences will be derived from those regions listed below or from any overlap regions between any of these peptides.

The sequences of predicted interest for containing potential antibody epitopes have been selected on the basis of less than 75% sequence identity.

| Region | Position | % sequence identity |
|---|---|---|
| i | 25-48 | 63% |
| ii | 49-75 | 74% |
| iii | 93-114 | 59% |
| iv | 123-139 | 63% |
| v | 158-176 | 68% |
| vi | 208-227 | 45% |
| vii | 231-248 | 21% |

VCAM-1

Human and porcine VCAM-1 protein sequences were aligned and regions of non-homology identified. We predict that the peptide sequences will be derived from those regions listed below or from any overlap regions between any of these peptides.

The sequences of predicted interest for containing potential antibody epitopes have been selected on the basis of less than 75% sequence identity.

| Region | Position | % sequence identity |
|---|---|---|
| i | 1-15 | 44% |
| ii | 16-33 | 63% |
| iii | 49-65 | 58% |
| iv | 74-85 | 42% |
| v | 100-117 | 50% |
| vi | 122-140 | 56% |
| vii | 144-157 | 64% |
| viii | 162-191 | 47% |
| ix | 209-221 | 62% |
| x | 290-301 | 67% |
| xi | 322-342 | 62% |
| xii | 362-379 | 67% |
| xiii | 448-465 | 67% |

1. A method of improving tolerance to a xenograft comprising: immunising a mammal with an immunogen comprising at least one T-cell epitope and at least one porcine polypeptide B-cell epitope, wherein said B-cell epitope is capable of mediating rejection of said xenograft.

2. A method according to claim 1, wherein said B-cell epitope is a peptide derived from at least one porcine polypeptide selected from the group of CD40, CD80, CD86 and VCAM.

3. A method according to claim 1, wherein said peptide is selected from at least one peptide represented in FIG. 22.

4. A method according to claim 1, wherein said peptide is selected from at least one peptide represented in FIG. 24.

5. A method according to claim 1, wherein said peptide is selected from at least one peptide represented in FIG. 26.

6. A method according to claim 1, wherein said T-cell epitope comprises a tetanus toxoid polypeptide.

7. A composition comprising an immunogen characterised in that said immunogen comprises at least one B-cell epitope and at least one T-cell epitope wherein said B-cell epitope comprises a porcine epitope involved in mediating xenograft rejection.

8. A composition according to claim 7, wherein said porcine epitope comprises a porcine polypeptide expressed by vascular endothelial cells of said xenograft.

9. A composition according to claim 7, wherein said B-cell epitope is selected from the group of CD40, CD86, CD80 and VCAM.

10. A composition according to claim 9, wherein said B-cell epitope comprises at least one peptide as represented in FIG. 22.

11. A composition according to claim 9, wherein said B-cell epitope comprises at least one peptide as represented in FIG. 24.

12. A composition according to claim 9, wherein said B-cell epitope comprises at least one peptide as represented in FIG. 26.

13. A composition according to claim 9, wherein said B-cell epitope comprises an extracellular domain of CD86.

14. A composition according to claim 7, wherein said T-cell epitope comprises a tetanus toxoid epitope.

15. A composition according to claim 7, wherein said composition further comprises a carrier capable of enhancing the immune response to said immunogen.

16. An antibody, or the effective part thereof, wherein said antibody is capable of distinguishing between porcine polypeptides according to claim 7, and the homologous polypeptides of the mammal receiving said xenograft.

17. An antibody according to claim 16, wherein said antibody is monoclonal.

18. An antibody according to claim 16, wherein said antibody is a modified antibody comprising at least one detectable label.

19. A method to monitor an immune status of a mammalian recipient of a xenograft comprising:
i) removing a sample from a xenograft recipient to be tested;
ii) contacting said sample to the antibody according to claim 16; and
iii) monitoring expression of a porcine polypeptide shown in FIGS. 22, 24, or 26.

20. A method of treating a mammal prior to receiving a xenograft, comprising:
i) immunising a mammal with an immunogenic composition according to claim 7;
ii) assessing an immune status of said mammal to said immunogenic composition;
iii) transplanting said xenograft tissue/organ into a recipient mammal; and
iv) monitoring a rejection response to said xenograft.

21. A method according to claim 20, wherein said xenograft is of porcine origin and said mammal is human.

22. A method according to claim 20, wherein said xenograft comprises at least one vascularised graft and/or immunogenic porcine cell/tissue.

23. A method according to claim 20, wherein said xenograft comprises pancreatic islets.

24. The method claim 1, wherein said B-cell epitope has less than 75% sequence identity to a corresponding region of an equivalent human polypeptide.

25. The method of claim 7, wherein said B-cell epitope has less than 75% sequence identity to a corresponding region of an equivalent human polypeptide.

26. The method of claim 16, wherein said B-cell epitope has less than 75% sequence identity to a corresponding region of an equivalent human polypeptide.
